(19) United States
(12) Patent Application Publication
(10) Pub. No.: US 2023/0365694 A1
(43) Pub. Date: Nov. 16, 2023

(54) IMMUNOSILENCING Fc VARIANTS

(71) Applicant: VISTERRA, INC., Waltham, MA (US)

(72) Inventor: Karthik VISWANATHAN, Waltham, MA (US)

(73) Assignee: VISTERRA, INC., Waltham, MA (US)

(21) Appl. No.: 18/052,131

(22) Filed: Nov. 2, 2022

Related U.S. Application Data
(60) Provisional application No. 63/274,716, filed on Nov. 2, 2021.

(51) Int. Cl. C07K 16/28

(52) U.S. Cl. CPC C07K 16/283; C07K 2317/52; C07K 2317/73

(57) ABSTRACT

The present disclosure provides, among other things Fc variants that have significantly reduced ADCC, ADCP and CDC function As described herein, the present disclosure is, in part based on identification of novel combinations of mutations that abolish binding to all FcγRI, FcγRIIa, FcγRIIb, FcγRIIIa, FcγRIIIb, and C1q.

### BACKGROUND

The immune response is a mechanism by which the body defends itself against foreign substances that invade it, causing infection or disease. This mechanism is based on the ability of antibodies produced or administered to the host to bind the antigen though its variable region. Once the antigen is bound by the antibody, the antigen is targeted for destruction, often mediated in part, by the constant region or Fc domain of the antibody.

For example, one activity of the Fc domain of the antibody is to bind complement proteins which can assist in lysing the target antigen, for example, a cellular pathogen. Another activity of the Fc region is to bind to Fc receptors (FcR) on the surface of immune cells, or so-called effector cells, which have the ability to trigger other immune effects. These immune effects include, for example, release of immune activators, regulation of antibody production, endocytosis, phagocytosis, and cell killing. In some clinical applications these responses are crucial for the efficacy of the antibody while in other cases they provoke unwanted side effects. One example of an effector-mediated side effect is the release of inflammatory cytokines causing an acute fever reaction. Another example is the long term deletion of antigen-bearing cells.

The effector function of an antibody can be avoided by using antibody fragments lacking the Fc region (e.g., such as a Fab, Fab′2, or single chain antibody (sFv)) however these fragments have a reduced half-life, only one antigen binding site instead of two (e.g., in the case of Fab antibody fragments and single chain antibodies (sFv)), and are more difficult to purify.

### SUMMARY OF THE INVENTION

The present disclosure provides, among other things, Fc variants that have significantly reduced ADCC, ADCP and CDC function. As described herein, the present disclosure is, in part based on identification of novel combinations of mutations that abolish binding to all FcγRI, FcγRIIa, FcγRIIb, FcγRIIIa, FcγRIIIb, and C1q, and maintain its ability to bind to FcRn.

In one aspect, the present invention provides, among other things, an isolated polypeptide comprising an Fc variant of a wild-type human IgG Fc region, said Fc variant comprising an amino acid substitutions at positions 235 and 265, wherein the amino acid at 265 is substituted to Gly, wherein the residues are numbered according to the EU index.

In some embodiments, an Fc variant further comprises one or more amino acid substitutions at 234, 237, 329, 330, or 331. In some embodiments, an Fc variant further comprises an amino acid substitution at 234. In some embodiments, an Fc variant further comprises amino acid substitutions at 234 and 237. In some embodiments, an Fc variant further comprises amino acid substitutions at 234, 330, and 331. In some embodiments, an Fc variant further comprises amino acid substitutions at 234, 237, 330, and 331. In some embodiments, an Fc variant further comprises an amino acid substitution at 237. In some embodiments, an Fc variant further comprises amino acid substitutions at 330 and 331. In some embodiments, an Fc variant further comprises an amino acid substitution at 329.

In one aspect, the present invention provides, among other things, an isolated polypeptide comprising an Fc variant of a wild-type human IgG Fc region, said Fc variant comprising an amino acid substitutions at positions 234 and 265, wherein the amino acid at 234 is substituted to Val, wherein the residues are numbered according to the EU index.

In some embodiments, an Fc variant further comprises amino acid substitutions at 235 and 237. In some embodiments, an Fc variant further comprises amino acid substitutions at 235, 237, 330 and 331. In some embodiments, an Fc variant further comprises an amino acid substitution at 235.

In one aspect, the present invention provides, among other things, an isolated polypeptide comprising an Fc variant of a wild-type human IgG4 Fc region, said Fc variant comprising an amino acid substitutions at positions F234, L235, and D265, wherein the residues are numbered according to the EU index.

In one aspect, the present invention provides, among other things, an isolated polypeptide comprising an Fc variant of a wild-type human IgG Fc region, said Fc variant comprising an amino acid substitutions of 234V, L235E, and D265G, wherein the residues are numbered according to the EU index.

In some embodiments, an Fc variant is IgG4 Fc region. In some embodiments, an Fc variant comprises amino acid substitutions of S228P, F234V, L235E, and D265G. In some embodiments, an Fc variant is IgG4 Fc region and comprises amino acid substitutions of S228P, F234V, L235E, and D265G.

In one aspect, the present invention provides, among other things, an isolated polypeptide comprising an Fc variant of a wild-type human IgG Fc region, said Fc variant comprising an amino acid substitutions of 234F, L235E, and D265G, wherein the residues are numbered according to the EU index.

In some embodiments, an Fc variant is IgG1 Fc region. In some embodiments, an Fc variant comprises amino acid substitutions of L234F, L235E, and D265G. In some embodiments, an Fc variant is IgG1 Fc region and comprises amino acid substitutions of L234F, L235E, and D265G.

In one aspect, the present invention provides, among other things, an isolated polypeptide comprising an Fc variant of a wild-type human IgG Fc region, said Fc variant comprising an amino acid substitutions of 234F, L235E, G237A, and D265G, wherein the residues are numbered according to the EU index.

In some embodiments, an Fc variant is IgG1 Fc region. In some embodiments, an Fc variant comprises amino acid substitutions of L234F, L235E, G237A, and D265G. In some embodiments, an Fc variant is IgG1 Fc region and comprises amino acid substitutions of L234F, L235E, G237A, and D265G.

In one aspect, the present invention provides, among other things, an isolated polypeptide comprising an Fc variant of a wild-type human IgG Fc region, said Fc variant comprising an amino acid substitutions of 234V, L235E, G237A, and D265G, wherein the residues are numbered according to the EU index.

In some embodiments, an Fc variant is IgG1 Fc region. In some embodiments, an Fc variant comprises amino acid substitutions of L234V, L235E, G237A, and D265G. In some embodiments, an Fc variant is IgG1 Fc region and comprises amino acid substitutions of L234V, L235E, G237A, and D265G.

In one aspect, the present invention provides, among other things, an isolated polypeptide comprising an Fc variant of a wild-type human IgG Fc region, said Fc variant comprising an amino acid substitutions of 234F, L235E, D265D, A330S, and P331S, wherein the residues are numbered according to the EU index.

In some embodiments, an Fc variant is IgG1 Fc region. In some embodiments, an Fc variant comprises amino acid substitutions of L234F, L235E, D265D, A330S, and P331S. In some embodiments, an Fc variant is IgG1 Fc region and comprises amino acid substitutions of L234F, L235E, D265D, A330S, and P331S.

In one aspect, the present invention provides, among other things, an isolated polypeptide comprising an Fc variant of a wild-type human IgG Fc region, said Fc variant comprising an amino acid substitutions of 234V, L235A, G237A, and D265G, wherein the residues are numbered according to the EU index.

In some embodiments, an Fc variant is IgG1 Fc region. In some embodiments, an Fc variant comprises amino acid substitutions of L234V, L235A, G237A, and D265G. In some embodiments, an Fc variant is IgG1 Fc region and comprises amino acid substitutions of 234F, L234V, L235A, G237A, and D265G. In some embodiments, an Fc variant is IgG4 Fc region. In some embodiments, an Fc variant comprises amino acid substitutions of S228P, L234V, L235A, G237A, and D265G. In some embodiments, an Fc variant is IgG4 Fc region and comprises amino acid substitutions of S228P, L234V, L235A, G237A, and D265G.

In one aspect, the present invention provides, among other things, an isolated polypeptide comprising an Fc variant of a wild-type human IgG Fc region, said Fc variant comprising an amino acid substitutions of 234V, L235A, G237A, D265G, A330S, and P331S wherein the residues are numbered according to the EU index.

In some embodiments, an Fc variant is IgG1 Fc region. In some embodiments, an Fc variant comprises amino acid substitutions of L234V, L235A, G237A, D265G, A330S, and P331S. In some embodiments, an Fc variant is IgG1 Fc region and comprises amino acid substitutions of L234V, L235A, G237A, D265G, A330S, and P331S.

In one aspect, the present invention provides, among other things, an isolated polypeptide comprising an Fc variant of a wild-type human IgG Fc region, said Fc variant comprising an amino acid substitutions of L235E and D265G, wherein the residues are numbered according to the EU index.

In some embodiments, an Fc variant is IgG4 Fc region. In some embodiments, an Fc variant comprises amino acid substitutions of S228P, L235E and D265G. In some embodiments, an Fc variant is IgG4 Fc region and comprises amino acid substitutions of S228P, L235E and D265G.

In one aspect, the present invention provides, among other things, an isolated polypeptide comprising an Fc variant of a wild-type human IgG Fc region, said Fc variant comprising an amino acid substitutions of L235E, G237A, and D265G, wherein the residues are numbered according to the EU index.

In some embodiments, an Fc variant is IgG4 Fc region. In some embodiments, an Fc variant comprises amino acid substitutions of S228P, L235E, G237A, and D265G. In some embodiments, an Fc variant is IgG4 Fc region and comprises amino acid substitutions of S228P, L235E, G237A, and D265G.

In one aspect, the present invention provides, among other things, an isolated polypeptide comprising an Fc variant of a wild-type human IgG Fc region, said Fc variant comprising an amino acid substitutions of L235E, G237A, and P329G, wherein the residues are numbered according to the EU index.

In some embodiments, an Fc variant is IgG4 Fc region. In some embodiments, an Fc variant comprises amino acid substitutions of S228P, L235E, G237A, and P329G. In some embodiments, an Fc variant is IgG4 Fc region and comprises amino acid substitutions of S228P, L235E, G237A, and P329G.

In one aspect, the present invention provides, among other things, an isolated polypeptide comprising an Fc variant of a wild-type human IgG Fc region, said Fc variant comprising an amino acid substitutions of L235E, G237A, and L328R, wherein the residues are numbered according to the EU index.

In some embodiments, an Fc variant is IgG4 Fc region. In some embodiments, an Fc variant comprises amino acid substitutions of S228P, L235E, G237A, and L328R. In some embodiments, an Fc variant is IgG4 Fc region and comprises amino acid substitutions of S228P, L235E, G237A, and L328R.

In one aspect, the present invention provides, among other things, an isolated polypeptide comprising an Fc variant of a wild-type human IgG Fc region, said Fc variant comprising an amino acid substitutions of D265G, A330S, and P331S, wherein the residues are numbered according to the EU index.

In some embodiments, an Fc variant is IgG2 Fc region. In some embodiments, an Fc variant comprises amino acid substitutions of D265G, A330S, and P331S. In some embodiments, an Fc variant is IgG2 Fc region and comprises amino acid substitutions of D265G, A330S, and P331S.

In one aspect, the present invention provides, among other things, an isolated polypeptide comprising an Fc variant of a wild-type human IgG Fc region, said Fc variant comprising an amino acid substitutions of 235E, D265G, A330S, and P331S, wherein the residues are numbered according to the EU index.

In some embodiments, an Fc variant is IgG2 Fc region. In some embodiments, an Fc variant comprises amino acid substitutions of A235E, D265G, A330S, and P331S. In some embodiments, an Fc variant is IgG2 Fc region and comprises amino acid substitutions of A235E, D265G, A330S, and P331S.

In one aspect, the present invention provides, among other things, an isolated polypeptide comprising an Fc variant of a wild-type human IgG Fc region, said Fc variant comprising an amino acid substitutions of 235E, D265G, and P329G, wherein the residues are numbered according to the EU index.

In some embodiments, an Fc variant is IgG2 Fc region. In some embodiments, an Fc variant comprises amino acid substitutions of A235E, D265G, and P329G. In some embodiments, an Fc variant is IgG2 Fc region and comprises amino acid substitutions of A235E, D265G, and P329G.

In one aspect, the present invention provides, among other things, a nucleic acid encoding an isolated polypeptide comprising an Fc variant of the present invention.

In one aspect, the present invention provides, among other things, a cell comprising a nucleic acid encoding an isolated polypeptide comprising an Fc variant of the present invention.

In one aspect, the present invention provides a method of treating a disease or disorder, said method comprising administering a therapeutically effective amount of an isolated polypeptide comprising an Fc variant to a subject in need of In some embodiments, the disease or disorder is ANCA-associated vasculitis. In some embodiments, the disease or disorder is C3 glomerulopathy (C3G). In some embodiments, the disease or disorder is an autoimmune disease. In some embodiments, the disease or disorder is wet age-related macular degeneration (wAMD). In some embodiments, the disease or disorder is Passive Heymann Nephritis (NHP). In some embodiments, the disease or disorder is collagen-antibody induced arthritis (CAIA).

### BRIEF DESCRIPTION OF FIGURES

FIGS. 1A-D are a series of exemplary graphs illustrating binding of Ab1 and Ab2 engineered with various Fc variants to FcγRI, FcγRIIa, FcγRIIb, and C1q, respectively, determined by the assay described in Example 2.

FIG. 2A is an exemplary bar graph and a table illustrating the expression level of the Ab1 engineered with wild-type IgG1, wild-type IgG4, vFc10, and vFc17. FIG. 2B is an exemplary graph and a table illustrating the protein A binding properties of the Ab 1 engineered with wild-type IgG1, wild-type IgG4, vFc17.

FIG. 3A is an exemplary graph illustrating binding of Ab 1 engineered with wild-type IgG1, wild-type IgG4, vFc10 or vFc17 to FcγRI, demonstrating that the Fc variants of the present invention significantly reduce binding of Fc to the FcγRI as compared to the wild-type IgG1 and IgG4. FIG. 3B is an exemplary graph illustrating binding of Ab 1 engineered with wild-type IgG1, wild-type IgG4, vFc10 or vFc17 to FcγRIIa and FcγRIIb, demonstrating that the Fc variants of the present invention significantly reduce binding of Fc to the FcγRIIa and FcγRIIb as compared to the wild-type IgG1 and IgG4. FIG. 3C is an exemplary graph illustrating binding of Ab1 engineered with wild-type IgG1, wild-type IgG4, vFc10 or vFc17 to

FcγRIIIa and FcγRIIIb. FIG. 3D is an exemplary graph illustrating binding of Ab1 engineered with wild-type IgG1, wild-type IgG4, vFc10 or vFc17 to C1q.

FIG. 4A is an exemplary graph illustrating fold induction of ADCC by Ab1 engineered with wild-type IgG1, wild-type IgG4, vFc10 or vFc17. FIG. 4B is an exemplary graph illustrating fold induction of ADCP by Ab 1 engineered with wild-type IgG1, wild-type IgG4, vFc10 or vFc17. FIG. 4C is an exemplary graph illustrating fold induction of CDC by Ab1 engineered with wild-type IgG1, wild-type IgG4, vFc10 or vFc17.

### DEFINITIONS

Antibody: As used herein, the term “antibody” refers to immunoglobulin molecules and immunologically active portions of immunoglobulin (Ig) molecules, i.e., molecules that contain an antigen binding site that binds (immunoreacts with) an antigen. By “binds” or “immunoreacts with” is meant that the antibody reacts with one or more antigenic determinants of the desired. Antibodies include, antibody fragments. Antibodies also include, but are not limited to, polyclonal, monoclonal, chimeric dAb (domain antibody), single chain, Fab, Fab′, F(ab′)2 fragments, scFvs, and Fab expression libraries. An antibody may be a whole antibody, or immunoglobulin, or an antibody fragment.

Fc domain: As used herein, the term “Fc region” refers to a C-terminal region of an immunoglobulin heavy chain that contains at least a portion of the constant region. The term includes native sequence Fc regions and variant Fc regions. In one embodiment, a human IgG heavy chain Fc region extends from Cys226, or from Pro230, to the carboxyl-terminus of the heavy chain. However, the C-terminal lysine (Lys447) of the Fc region may or may not be present. Unless otherwise specified herein, numbering of amino acid residues in the Fc region or constant region is according to the EU numbering system, also called the EU index, as described in Kabat et al., Sequences of Proteins of Immunological Interest, 5th Ed. Public Health Service, National Institutes of Health, Bethesda, Md., 1991.

Fab arm exchange: The term, “Fab arm exchange” refers to the phenomenon that IgG4 antibodies can exchange ‘half-molecules’, an activity termed Fab arm exchange herein. Especially in bispecific or biparatopic molecules, this results in functionally monovalent antibodies with unknown specificity and hence, potentially, reduced therapeutic efficacy. Mutations can be introduced in the Fc domain to inhibit the Fab arm exchange. It is known that S228P mutation can prevent IgG4 FAE to undetectable levels both in vitro and in vivo.

Humanized antibody: The term “humanized antibody” includes non-human (e.g., murine) antibodies that are specific immunoglobulin chains, chimeric immunoglobulins, or fragments thereof that contain minimal non-human (e.g., murine) sequences. Typically, humanized antibodies are human immunoglobulins in which residues from the complementary determining region (CDR) are replaced by residues from the CDR of a non-human species (e.g., mouse, rat, rabbit, hamster) that have the desired specificity, affinity, and capability (Jones et al., Nature 321:522-525, 1986; Riechmann et al., Nature 332:323-327, 1988; Verhoeyen et al., Science 239:1534-1536, 1988).

Monoclonal Antibody: The term “monoclonal antibody” refers to an antibody obtained from a population of substantially homogeneous antibodies, i.e., the individual antibodies comprising the population are identical except for possible naturally-occurring mutations that may be present in minor amounts. Monoclonal antibodies are highly specific, being directed against a single antigenic site. The modifier “monoclonal” indicates the character of the antibody as being obtained from a substantially homogeneous population of antibodies, and is not to be construed as requiring production of the antibody by any particular method.

Multispecific antibody: As used herein, the term “multispecific antibody” refers to binding molecules, antibodies, or antigen-binding fragments thereof that have the ability to specifically bind to two or more different epitopes on the same or different target(s).

Biparatopic antibody: As used herein, the term “biparatopic antibody” refers to a multispecific antibody having the capability of binding 2 different non-overlapping epitopes on the same target antigen molecule.

Kᵢ or K_{d}: As used herein, the term “K_{d}”, as used herein, refers to the dissociation constant of a particular antibody-antigen interaction as is known in the art, and would apply as a parameter of the binding affinity of a targeting moiety to its cognate ligand for the subject compositions.

IC50: As used herein, the term “IC50” refers to the concentration needed to inhibit half of the maximum biological response of the ligand agonist, and is generally determined by competition binding assays.

EC50: As used herein, the term “EC50” refers to a half maximal effective concentration. The term EC50 refers to the concentration of a drug, antibody or toxicant which induces a response halfway between the baseline and maximum after a specified exposure time. More simply, EC50 can be defined as the concentration required to obtain a 50% of the desired effect.

Linker: As used herein, the term “linker” refers to a molecule or group of molecules (such as a monomer or polymer) that connects two molecules and often serves to place the two molecules in a preferred configuration. A number of strategies may be used to covalently link molecules together. These include, but are not limited to polypeptide linkages between N- and C-terminus of proteins or protein domains, linkage via disulfide bonds, and linkage via chemical cross-linking reagents. In one aspect of this embodiment, the linker is a peptide bond, generated by recombinant techniques or peptide synthesis. The linker may contain amino acid residues that provide flexibility. Thus, the linker peptide may predominantly include the following amino acid residues: Gly, Ser, Ala, or Thr. The linker peptide should have a length that is adequate to link two molecules in such a way that they assume the correct conformation relative to one another so that they retain the desired activity. Suitable lengths for this purpose include at least one and not more than 30 amino acid residues. In one embodiment, the linker is from about 1 to 30 amino acids in length. In another embodiment, the linker is from about 1 to 15 amino acids in length. In addition, the amino acid residues selected for inclusion in the linker peptide should exhibit properties that do not interfere significantly with the activity of the polypeptide.

scFv: As used herein, the term “scFv” refers to a fusion protein of the variable regions of the heavy (VH) and light chains (VL) of immunoglobulins, connected with a short linker peptide of ten to about 25 amino acids.

Fab: As used herein, the term “Fab” refers to an antibody fragment comprising a portion of an intact antibody, comprising the antigen-binding or variable region thereof.

In vitro: As used herein, the term “in vitro” refers to events that occur in an artificial environment, e.g., in a test tube or reaction vessel, in cell culture, etc., rather than within a multi-cellular organism.

In vivo: As used herein, the term “in vivo” refers to events that occur within a multi-cellular organism, such as a human and a non-human animal. In the context of cell-based systems, the term may be used to refer to events that occur within a living cell (as opposed to, for example, in vitro systems).

Subject: As used herein, the term “subject” refer to a human or any non-human animal (e.g., mouse, rat, rabbit, dog, cat, cattle, swine, sheep, horse or primate). A human includes pre- and post-natal forms. In many embodiments, a subject is a human being. A subject can be a patient, which refers to a human presenting to a medical provider for diagnosis or treatment of a disease. The term “subject” is used herein interchangeably with “individual” or “patient.” A subject can be afflicted with or is susceptible to a disease or disorder but may or may not display symptoms of the disease or disorder.

Dysfunction: As used herein, the term “dysfunction” refers to an abnormal function. A dysfunction of a molecule (e.g., a protein) can be caused by an increase or decrease in activity associated with such molecule. A dysfunction of a molecule can be caused by a defect associated with the molecule itself, or other molecules that interact directly or indirectly with or regulate the molecule.

Derivatives: As used herein, the term “derivatives” when used in connection with antibody, or C5aR1 antibodies, refer to a portion having some of the sequence of an original molecule that retains at least some of the functions and/or properties of the original molecule.

Identity: As used herein, the term “identity” refers a relationship between the sequences of two or more polypeptide molecules or two or more nucleic acid molecules as known in the art, comparing the sequences of these molecules. The relationship determined by doing. In the art, “identity” also means the degree of sequence relatedness between nucleic acid molecules or polypeptides, and in some cases more than one nucleotide sequence or more than one. It may be determined by a match between amino acid sequence strings. “Identity” means between a gap alignment (if any) addressed by a particular mathematical model or computer program (ie, an “algorithm”) and a smaller sequence of two or more sequences. Measure the percent identity match.

Similarity or Similar: As used herein, the term “similarity” is used in the art with respect to related concepts, but in contrast to “identity,” “similarity”, refers to both identity and conservative substitution matches. Indicating relevance, including if two polypeptide sequences have, for example, 10 identical amino acids out of 20 amino acids and the rest are all non-conservative substitutions, the percent identity and percent similarity are both 50%. In the same example, if there are 5 more conservative substitutions, the percent identity remains 50%, but the percent similarity is 75%. Thus, if there are conservative substitutions, the percent similarity between the two polypeptides is higher than the percent identity between these two polypeptides.

Treating: As used herein, the term “treat,” “treatment,” or “treating” refers to any method used to partially or completely alleviate, ameliorate, relieve, inhibit, prevent, delay onset of, reduce severity of and/or reduce incidence of one or more symptoms or features of a particular disease, disorder, and/or condition. Treatment may be administered to a subject who does not exhibit signs of a disease and/or exhibits only early signs of the disease for the purpose of decreasing the risk of developing pathology associated with the disease.

Vector: The term “vector” refers to a polynucleotide (usually DNA) used to artificially carry foreign genetic material to another cell where it can be replicated or expressed. Non-limiting exemplary vectors include plasmids, viral vectors, cosmids, and artificial chromosomes. Such vectors may be derived from a variety of sources, including bacterial and viral sources. A non-limiting exemplary viral source for a plasmid is adeno-associated virus.

Various aspects of the disclosure are described in detail in the following sections. The use of sections is not meant to limit the disclosure. Each section can apply to any aspect of the disclosure. In this application, the use of “or” means “and/or” unless stated otherwise. As used herein, the singular forms “a”, “an”, and “the” include both singular and plural referents unless the context clearly dictates otherwise.

### DETAILED DESCRIPTION

The Fc region of an antibody controls antibody cytotoxic activities and can impact serum half-life of the antibody. In a therapeutic context, however, the cytotoxic effector function of an antibody is often not desirable and can create safety concerns and unwanted side effects by activating host immune defenses. In cases where additional activation is detrimental, Fc-engineering is required to silence the IgG Fc domain such that it cannot bind to Fc-Gamma receptors.

The present disclosure describes new classes of Fc variants that are particularly effective in silencing the IgG Fc domain. Notably, the combinations Fc mutations of the present invention are novel and reduce binging to the Fc-Gamma receptors and C1q, effectively reducing the undesired ADCC, ADCP, and CDC effector functions.

Fc Region and Its Effector Functions

The Fc region of an antibody interacts with a number of Fc receptors and ligands, imparting an array of important functional capabilities referred to as effector functions. For IgG, the Fc region comprises Ig domains Cγ2 and Cγ3 and the N-terminal hinge leading into Cγ2. An important family of Fc receptors for the IgG class are the Fc gamma receptors (FcγRs). These receptors mediate communication between antibodies and the cellular arm of the immune system (Raghavan et al., 1996, Annu Rev Cell Dev Biol 12:181-220; Ravetch et al., 2001, Annu Rev Immunol 19:275-290). In humans this protein family includes FcγRI (CD64), including isoforms FcγRIa, FcγRIb, and FcγRIc; FcγRII (CD32), including isoforms FcγRIIa (including allotypes H131 and R131), FcγRIIb (including FcγRIIb-1 and FcγRIIb-2), and FcγRIIc; and FcγRIII (CD16), including isoforms FcγRIIIa (including allotypes V158 and F158) and FcγRIIIb (including allotypes FcγRIIIb-NA1 and FcγRIIIb-NA2) (Jefferis et al., 2002, Immunol Lett 82:57-65, incorporated by reference). These receptors typically have an extracellular domain that mediates binding to Fc, a membrane spanning region, and an intracellular domain that may mediate some signaling event within the cell. These receptors are expressed in a variety of immune cells including monocytes, macrophages, neutrophils, dendritic cells, eosinophils, mast cells, platelets, B cells, large granular lymphocytes, Langerhans' cells, natural killer (NK) cells, and γδ T cells. Formation of the Fc/FcγR complex recruits these effector cells to sites of bound antigen, typically resulting in signaling events within the cells and important subsequent immune responses such as release of inflammation mediators, B cell activation, endocytosis, phagocytosis, and cytotoxic attack. The ability to mediate cytotoxic and phagocytic effector functions is a potential mechanism by which antibodies destroy targeted cells. The cell-mediated reaction wherein nonspecific cytotoxic cells that express FcγRs recognize bound antibody on a target cell and subsequently cause lysis of the target cell is referred to as antibody dependent cell-mediated cytotoxicity (ADCC) (Raghavan et al., 1996, Annu Rev Cell Dev Biol 12:181-220; Ghetie et al., 2000, Annu Rev Immunol 18:739-766; Ravetch et al., 2001, Annu Rev Immunol 19:275-290, incorporated by reference). The cell-mediated reaction wherein nonspecific cytotoxic cells that express FcγRs recognize bound antibody on a target cell and subsequently cause phagocytosis of the target cell is referred to as antibody dependent cell-mediated phagocytosis (ADCP). A number of structures have been solved of the extracellular domains of human FcγRs, including FcγRIIa (pdb accession code 1H9V) (Sondermann et al., 2001, J Mol Biol 309:737-749) (pdb accession code 1 FCG) (Maxwell et al., 1999, Nat Struct Biol 6:437-442), FcγRIIb (pdb accession code 2FCB) (Sondermann et al., 1999, Embo J 18:1095-1103); and FcγRIIIb (pdb accession code 1E4J) (Sondermann et al., 2000, Nature 406:267-273, incorporated by reference). All FcγRs bind the same region on Fc, at the N-terminal end of the Cγ2 domain and the preceding hinge. This interaction is well characterized structurally (Sondermann et al., 2001, J Mol Biol 309:737-749 incorporated by reference), and several structures of the human Fc bound to the extracellular domain of human FcγRIIIb have been solved (pdb accession code 1E4K)(Sondermann et al., 2000, Nature 406:267-273) (pdb accession codes 1IIS and 1IIX) (Radaev et al., 2001, J Biol Chem 276:16469-16477, incorporated by reference), as well as has the structure of the human IgE Fd/FccRla complex (pdb accession code 1F6A) (Garman et al., 2000, Nature 406:259-266, incorporated by reference).

An overlapping but separate site on Fc serves as the interface for the complement protein C1q. In the same way that Fc/FcγR binding mediates ADCC, Fc/C1q binding mediates complement dependent cytotoxicity (CDC). C1q forms a complex with the serine proteases C1r and C1s to form the C1 complex. C1q is capable of binding six antibodies, although binding to two IgGs is sufficient to activate the complement cascade. Similar to Fc interaction with FcγRs, different IgG subclasses have different affinity for C1q, with IgG1 and IgG3 typically binding substantially better to the FcγRs than IgG2 and IgG4.

A site on Fc between the Cγ2 and Cγ3 domains mediates interaction with the neonatal receptor FcRn, the binding of which recycles endocytosed antibody from the endosome back to the bloodstream (Raghavan et al., 1996, Annu Rev Cell Dev Biol 12:181-220; Ghetie et al., 2000, Annu Rev Immunol 18:739-766, incorporated by reference). This process, coupled with preclusion of kidney filtration due to the large size of the full length molecule, results in favorable antibody serum half-lives ranging from one to three weeks. Binding of Fc to FcRn also plays a key role in antibody transport. The binding site for FcRn on Fc is also the site at which the bacterial proteins A and G bind. The tight binding by these proteins is typically exploited as a means to purify antibodies by employing protein A or protein G affinity chromatography during protein purification. Thus the fidelity of this region on Fc is important for both the clinical properties of antibodies and their purification. Available structures of the rat Fc/FcRn complex (Martin et al., 2001, Mol Cell 7:867-877, incorporated by reference), and of the complexes of Fc with proteins A and G (Deisenhofer, 1981, Biochemistry 20:2361-2370; Sauer-Eriksson et al., 1995, Structure 3:265-278; Tashiro et al., 1995, Curr Opin Struct Biol 5:471-481, incorporated by reference) provide insight into the interaction of Fc with these proteins.

The present invention is directed to optimized Fc variants useful in a variety of contexts. As outlined above, current antibody therapies suffer from a variety of problems. The present invention provides a promising means for enhancing the therapeutic efficacy of antibodies is via abolishment of their ability to mediate cytotoxic effector functions such as ADCC, ADCP, and CDC.

Fc Variants of the Present Invention

The Fc variants of the present invention may find use in a variety of Fc polypeptides. An Fc polypeptide that comprises an Fc variant of the present invention is herein referred to as an “Fc polypeptide of the present invention”. Fc polypeptides of the present invention include polypeptides that comprise the Fc variants of the present invention in the context of a larger polypeptide, such as an antibody or Fc fusion. That is, Fc polypeptides of the present invention include antibodies and Fc fusions that comprise Fc variants of the present invention. By “antibody of the present invention” as used herein is meant an antibody that comprises an Fc variant of the present invention. By “Fc fusion of the present invention” as used herein refers to an Fc fusion that comprises an Fc variant of the present invention. Fc polypeptides of the present invention also include polypeptides that comprise little or no additional polypeptide sequence other than the Fc region, referred to as an isolated Fc. Fc polypeptides of the present invention also include fragments of the Fc region. As described below, any of the aforementioned Fc polypeptides of the present invention may be fused to one or more fusion partners or conjugate partners to provide desired functional properties.

The parent Fc polypeptides described herein may be derived from a wide range of sources, and may be substantially encoded by one or more Fc genes from any organism, including but not limited to humans, rodents including but not limited to mice and rats, lagomorpha such as rabbits and hares, camelidae such as camels, llamas, and dromedaries, and non-human primates, including but not limited to Prosimians, Platyrrhini (New World monkeys), Cercopithecoidea (Old World monkeys), and Hominoidea include the Gibbons, Lesser and Great Apes, with humans most preferred. The parent Fc polypeptides of the present invention may be substantially encoded by immunoglobulin genes belonging to any of the antibody classes, including but not limited to sequences belonging to the IgG (including human subclasses IgG1, IgG2, IgG3, or IgG4), IgA (including human subclasses IgA1 and IgA2), IgD, IgE, IgG, or IgM classes of antibodies. The parent Fc polypeptides of the present invention comprise sequences belonging to the human IgG class of antibodies. For example, the parent Fc polypeptide may be a parent antibody, for example a human IgG1 antibody, a human IgA antibody, or a mouse IgG2a or IgG2b antibody. Said parent antibody may be nonhuman, chimeric, humanized, or fully human as described in detail below. The parent Fc polypeptide may be modified or engineered in some way, for example a parent antibody may be affinity matured, or may possess engineered glycoforms, all as described more fully below. Alternatively, the parent Fc polypeptide may be an Fc fusion, for example an Fc fusion wherein the fusion partner targets a cell surface receptor. Alternatively, the parent Fc polypeptide may be an isolated Fc region, comprising little or no other polypeptide sequence outside the Fc region. The parent Fc polypeptide may be a naturally existing Fc region, or may be an existing engineered variant of an Fc polypeptide. What is important is that the parent Fc polypeptide comprise an Fc region, which can then be mutated to generate an Fc variant.

The Fc variants of the present invention may find use in a wide range of products. In one embodiment the Fc variant of the invention is a therapeutic, a diagnostic, or a research reagent, preferably a therapeutic. Alternatively, the Fc variant of the present invention may be used for agricultural or industrial uses. An antibody of the present invention may find use in an antibody composition that is monoclonal or polyclonal. The Fc variants of the present invention may be agonists, antagonists, neutralizing, inhibitory, or stimulatory. In a preferred embodiment, the Fc variants of the present invention are used to kill target cells that bear the target antigen, for example cancer cells. In an alternate embodiment, the Fc variants of the present invention are used to block, antagonize, or agonize the target antigen. In an alternately preferred embodiment, the Fc variants of the present invention are used to block, antagonize, or agonize the target antigen and kill the target cells that bear the target antigen.

Optimized Properties

The present invention provides Fc variants that are optimized for a number of therapeutically relevant properties. An Fc variant comprises one or more amino acid modifications relative to a parent Fc polypeptide, wherein said amino acid modification(s) provide one or more optimized properties. An Fc variant of the present invention differs in amino acid sequence from its parent Fc polypeptide by virtue of at least one amino acid modification. Thus Fc variants of the present invention have at least one amino acid modification compared to the parent. Alternatively, the Fc variants of the present invention may have more than one amino acid modification as compared to the parent, for example from about one to fifty amino acid modifications, from about one to ten amino acid modifications, or from about one to about five amino acid modifications compared to the parent. Thus the sequences of the Fc variants and those of the parent Fc polypeptide are substantially homologous. For example, the variant Fc variant sequences herein will possess about 80% homology with the parent Fc variant sequence, preferably at least about 90% homology, and most preferably at least about 95% homology.

The Fc variants of the present invention may be optimized for a variety of properties. An Fc variant that is engineered or predicted to display one or more optimized properties is herein referred to as an “optimized Fc variant”. Properties that may be optimized include but are not limited to enhanced or reduced affinity for an FcγR. In some embodiments, the Fc variants of the present invention are optimized to have reduced or ablated affinity for a human FcγR, including but not limited to FcγRI, FcγRIIa, FcγRIIb, FcγRIIc, FcγRIIIa, and FcγRIIIb. These embodiments are anticipated to provide Fc polypeptides with enhanced therapeutic properties in humans, for example reduced effector function and reduced toxicity. In other embodiments, Fc variants of the present invention provide enhanced affinity for one or more FcγRs, yet reduced affinity for one or more other FcγRs. For example, an Fc variant of the present invention may have enhanced binding to FcγRIIIa, yet reduced binding to FcγRIIb. Alternately, an Fc variant of the present invention may have enhanced binding to FcγRIIa and FcγRI, yet reduced binding to FcγRIIb. In yet another embodiment, an Fc variant of the present invention may have enhanced affinity for FcγRIIb, yet reduced affinity to one or more activating FcγRs.

In some embodiments, an Fc variant has reduced or ablated affinity for FcγRI. In some embodiments, an Fc variant has reduced or ablated affinity for FcγRIIa. In some embodiments, an Fc variant has reduced or ablated affinity for FcγRIIb. In some embodiments, an Fc variant has reduced or ablated affinity for FcγRIIc. In some embodiments, an Fc variant has reduced or ablated affinity for FcγRIIIa. In some embodiments, an Fc variant has reduced or ablated affinity for FcγRIIIb. In some embodiments, an Fc variant has reduced or ablated affinity for C1q. In some embodiments, an Fc variant has enhanced affinity for FcRn. In some embodiments, an Fc variant maintains affinity for FcRn. In some embodiments, an Fc variant has reduced or ablated affinity for FcγRI, FcγRIIa, FcγRIIb, FcγRIIIa, FcγRIIIb, and C1q. In some embodiments, an Fc variant has reduced or ablated affinity for FcγRI, FcγRIIa, FcγRIIb, FcγRIIIa, FcγRIIIb, and C1q, and retains binding to FcRn.

The Fc variants of the present invention may also be optimized for enhanced functionality and/or solution properties in aglycosylated form. In a preferred embodiment, the aglycosylated Fc variants of the present invention bind an Fc ligand with reduced affinity than the aglycosylated form of the parent Fc variant. Said Fc ligands include but are not limited to FcγRs, C1q, FcRn, and proteins A and G, and may be from any source including but not limited to human, mouse, rat, rabbit, or monkey, preferably human. In an alternately preferred embodiment, the Fc variants are optimized to be more stable and/or more soluble than the aglycosylated form of the parent Fc variant.

Engineered Fc Mutations

In one aspect, the present invention provides, among other things, an isolated polypeptide comprising an Fc variant of a wild-type human IgG1 Fc region, said Fc variant comprising amino acid substitutions L234F/L235E/D265G, wherein the residues are numbered according to the EU index.

In one aspect, the present invention provides, among other things, an isolated polypeptide comprising an Fc variant of a wild-type human IgG1 Fc region, said Fc variant comprising amino acid substitutions L234F/L235E/G237A/D265G, wherein the residues are numbered according to the EU index.

In one aspect, the present invention provides, among other things, an isolated polypeptide comprising an Fc variant of a wild-type human IgG1 Fc region, said Fc variant comprising amino acid substitutions L234V/L235E/G237A/D265G, wherein the residues are numbered according to the EU index.

In one aspect, the present invention provides, among other things, an isolated polypeptide comprising an Fc variant of a wild-type human IgG1 Fc region, said Fc variant comprising amino acid substitutions L234F/L235E/D265G/A330S/P331S, wherein the residues are numbered according to the EU index.

In one aspect, the present invention provides, among other things, an isolated polypeptide comprising an Fc variant of a wild-type human IgG1 Fc region, said Fc variant comprising amino acid substitutions L234V/L235A/G237A/D265G, wherein the residues are numbered according to the EU index.

In one aspect, the present invention provides, among other things, an isolated polypeptide comprising an Fc variant of a wild-type human IgG1 Fc region, said Fc variant comprising amino acid substitutions L234V/L235A/G237A/D265G/A330S/P331S wherein the residues are numbered according to the EU index.

In one aspect, the present invention provides, among other things, an isolated polypeptide comprising an Fc variant of a wild-type human IgG1 Fc region, said Fc variant comprising an amino acid substitution D265G, wherein the residues are numbered according to the EU index.

In one aspect, the present invention provides, among other things, an isolated polypeptide comprising an Fc variant of a wild-type human IgG4 Fc region, said Fc variant comprising amino acid substitutions L235E/D265G/S228P wherein the residues are numbered according to the EU index.

In one aspect, the present invention provides, among other things, an isolated polypeptide comprising an Fc variant of a wild-type human IgG4 Fc region, said Fc variant comprising amino acid substitutions F234V/L235E/D265G/S228P wherein the residues are numbered according to the EU index.

In one aspect, the present invention provides, among other things, an isolated polypeptide comprising an Fc variant of a wild-type human IgG4 Fc region, said Fc variant comprising amino acid substitutions F234V/L235A/G237A/D265G/S228P wherein the residues are numbered according to the EU index.

In one aspect, the present invention provides, among other things, an isolated polypeptide comprising an Fc variant of a wild-type human IgG4 Fc region, said Fc variant comprising amino acid substitutions L235E/G237A/D265G/S228P wherein the residues are numbered according to the EU index.

In one aspect, the present invention provides, among other things, an isolated polypeptide comprising an Fc variant of a wild-type human IgG4 Fc region, said Fc variant comprising amino acid substitutions L235E/G237A/P329G/S228P wherein the residues are numbered according to the EU index.

In one aspect, the present invention provides, among other things, an isolated polypeptide comprising an Fc variant of a wild-type human IgG4 Fc region, said Fc variant comprising amino acid substitutions L235E/G237A/L328R/S228P wherein the residues are numbered according to the EU index.

In one aspect, the present invention provides, among other things, an isolated polypeptide comprising an Fc variant of a wild-type human IgG2 Fc region, said Fc variant comprising amino acid substitutions D265G/A330S/P331S wherein the residues are numbered according to the EU index.

In one aspect, the present invention provides, among other things, an isolated polypeptide comprising an Fc variant of a wild-type human IgG2 Fc region, said Fc variant comprising amino acid substitutions A235E/D265G/A330S/P331S wherein the residues are numbered according to the EU index.

In one aspect, the present invention provides, among other things, an isolated polypeptide comprising an Fc variant of a wild-type human IgG2 Fc region, said Fc variant comprising amino acid substitutions A235E/D265G/P329G wherein the residues are numbered according to the EU index.

In one aspect, the present invention provides, among other things, an isolated polypeptide comprising an Fc variant of a wild-type human IgG Fc region, said Fc variant comprising an amino acid substitutions at positions 235 and 265, wherein the amino acid at 265 is substituted to Gly, wherein the residues are numbered according to the EU index.

In some embodiments, an Fc variant further comprises one or more amino acid substitutions at 234, 237, 329, 330, or 331. In some embodiments, an Fc variant further comprises an amino acid substitution at 234. In some embodiments, an Fc variant further comprises amino acid substitutions at 234 and 237. In some embodiments, an Fc variant further comprises amino acid substitutions at 234, 330, and 331. In some embodiments, an Fc variant further comprises amino acid substitutions at 234, 237, 330, and 331. In some embodiments, an Fc variant further comprises an amino acid substitution at 237. In some embodiments, an Fc variant further comprises amino acid substitutions at 330 and 331. In some embodiments, an Fc variant further comprises an amino acid substitution at 329.

In one aspect, the present invention provides, among other things, an isolated polypeptide comprising an Fc variant of a wild-type human IgG Fc region, said Fc variant comprising an amino acid substitutions at positions 234 and 265, wherein the amino acid at 234 is substituted to Val, wherein the residues are numbered according to the EU index.

In some embodiments, an Fc variant further comprises amino acid substitutions at 235 and 237. In some embodiments, an Fc variant further comprises amino acid substitutions at 235, 237, 330 and 331. In some embodiments, an Fc variant further comprises an amino acid substitution at 235.

In one aspect, the present invention provides, among other things, an isolated polypeptide comprising an Fc variant of a wild-type human IgG4 Fc region, said Fc variant comprising an amino acid substitutions at positions F234, L235, and D265, wherein the residues are numbered according to the EU index.

In one aspect, the present invention provides, among other things, an isolated polypeptide comprising an Fc variant of a wild-type human IgG Fc region, said Fc variant comprising an amino acid substitutions of 234V, L235E, and D265G, wherein the residues are numbered according to the EU index.

In some embodiments, an Fc variant is IgG4 Fc region. In some embodiments, an Fc variant comprises amino acid substitutions of S228P, F234V, L235E, and D265G. In some embodiments, an Fc variant is IgG4 Fc region and comprises amino acid substitutions of S228P, F234V, L235E, and D265G.

In one aspect, the present invention provides, among other things, an isolated polypeptide comprising an Fc variant of a wild-type human IgG Fc region, said Fc variant comprising an amino acid substitutions of 234F, L235E, and D265G, wherein the residues are numbered according to the EU index.

In some embodiments, an Fc variant is IgG1 Fc region. In some embodiments, an Fc variant comprises amino acid substitutions of L234F, L235E, and D265G. In some embodiments, an Fc variant is IgG1 Fc region and comprises amino acid substitutions of L234F, L235E, and D265G.

In one aspect, the present invention provides, among other things, an isolated polypeptide comprising an Fc variant of a wild-type human IgG Fc region, said Fc variant comprising an amino acid substitutions of 234F, L235E, G237A, and D265G, wherein the residues are numbered according to the EU index.

In some embodiments, an Fc variant is IgG1 Fc region. In some embodiments, an Fc variant comprises amino acid substitutions of L234F, L235E, G237A, and D265G. In some embodiments, an Fc variant is IgG1 Fc region and comprises amino acid substitutions of L234F, L235E, G237A, and D265G.

In one aspect, the present invention provides, among other things, an isolated polypeptide comprising an Fc variant of a wild-type human IgG Fc region, said Fc variant comprising an amino acid substitutions of 234V, L235E, G237A, and D265G, wherein the residues are numbered according to the EU index.

In some embodiments, an Fc variant is IgG1 Fc region. In some embodiments, an Fc variant comprises amino acid substitutions of L234V, L235E, G237A, and D265G. In some embodiments, an Fc variant is IgG1 Fc region and comprises amino acid substitutions of L234V, L235E, G237A, and D265G.

In one aspect, the present invention provides, among other things, an isolated polypeptide comprising an Fc variant of a wild-type human IgG Fc region, said Fc variant comprising an amino acid substitutions of 234F, L235E, D265D, A330S, and P331S, wherein the residues are numbered according to the EU index.

In some embodiments, an Fc variant is IgG1 Fc region. In some embodiments, an Fc variant comprises amino acid substitutions of L234F, L235E, D265D, A330S, and P331S. In some embodiments, an Fc variant is IgG1 Fc region and comprises amino acid substitutions of L234F, L235E, D265D, A330S, and P331S.

In one aspect, the present invention provides, among other things, an isolated polypeptide comprising an Fc variant of a wild-type human IgG Fc region, said Fc variant comprising an amino acid substitutions of 234V, L235A, G237A, and D265G, wherein the residues are numbered according to the EU index.

In some embodiments, an Fc variant is IgG1 Fc region. In some embodiments, an Fc variant comprises amino acid substitutions of L234V, L235A, G237A, and D265G. In some embodiments, an Fc variant is IgG1 Fc region and comprises amino acid substitutions of 234F, L234V, L235A, G237A, and D265G. In some embodiments, an Fc variant is IgG4 Fc region. In some embodiments, an Fc variant comprises amino acid substitutions of S228P, L234V, L235A, G237A, and D265G. In some embodiments, an Fc variant is IgG4 Fc region and comprises amino acid substitutions of S228P, L234V, L235A, G237A, and D265G.

In one aspect, the present invention provides, among other things, an isolated polypeptide comprising an Fc variant of a wild-type human IgG Fc region, said Fc variant comprising an amino acid substitutions of 234V, L235A, G237A, D265G, A330S, and P331S wherein the residues are numbered according to the EU index.

In some embodiments, an Fc variant is IgG1 Fc region. In some embodiments, an Fc variant comprises amino acid substitutions of L234V, L235A, G237A, D265G, A330S, and P331S. In some embodiments, an Fc variant is IgG1 Fc region and comprises amino acid substitutions of L234V, L235A, G237A, D265G, A330S, and P331S.

In one aspect, the present invention provides, among other things, an isolated polypeptide comprising an Fc variant of a wild-type human IgG Fc region, said Fc variant comprising an amino acid substitutions of L235E and D265G, wherein the residues are numbered according to the EU index.

In some embodiments, an Fc variant is IgG4 Fc region. In some embodiments, an Fc variant comprises amino acid substitutions of S228P, L235E and D265G. In some embodiments, an Fc variant is IgG4 Fc region and comprises amino acid substitutions of S228P, L235E and D265G.

In one aspect, the present invention provides, among other things, an isolated polypeptide comprising an Fc variant of a wild-type human IgG Fc region, said Fc variant comprising an amino acid substitutions of L235E, G237A, and D265G, wherein the residues are numbered according to the EU index.

In some embodiments, an Fc variant is IgG4 Fc region. In some embodiments, an Fc variant comprises amino acid substitutions of S228P, L235E, G237A, and D265G. In some embodiments, an Fc variant is IgG4 Fc region and comprises amino acid substitutions of S228P, L235E, G237A, and D265G.

In one aspect, the present invention provides, among other things, an isolated polypeptide comprising an Fc variant of a wild-type human IgG Fc region, said Fc variant comprising an amino acid substitutions of L235E, G237A, and P329G, wherein the residues are numbered according to the EU index.

In some embodiments, an Fc variant is IgG4 Fc region. In some embodiments, an Fc variant comprises amino acid substitutions of S228P, L235E, G237A, and P329G. In some embodiments, an Fc variant is IgG4 Fc region and comprises amino acid substitutions of S228P, L235E, G237A, and P329G.

In one aspect, the present invention provides, among other things, an isolated polypeptide comprising an Fc variant of a wild-type human IgG Fc region, said Fc variant comprising an amino acid substitutions of L235E, G237A, and L328R, wherein the residues are numbered according to the EU index.

In some embodiments, an Fc variant is IgG4 Fc region. In some embodiments, an Fc variant comprises amino acid substitutions of S228P, L235E, G237A, and L328R. In some embodiments, an Fc variant is IgG4 Fc region and comprises amino acid substitutions of S228P, L235E, G237A, and L328R.

In one aspect, the present invention provides, among other things, an isolated polypeptide comprising an Fc variant of a wild-type human IgG Fc region, said Fc variant comprising an amino acid substitutions of D265G, A330S, and P331S, wherein the residues are numbered according to the EU index.

In some embodiments, an Fc variant is IgG2 Fc region. In some embodiments, an Fc variant comprises amino acid substitutions of D265G, A330S, and P331S. In some embodiments, an Fc variant is IgG2 Fc region and comprises amino acid substitutions of D265G, A330S, and P331S.

In one aspect, the present invention provides, among other things, an isolated polypeptide comprising an Fc variant of a wild-type human IgG Fc region, said Fc variant comprising an amino acid substitutions of 235E, D265G, A330S, and P331S, wherein the residues are numbered according to the EU index.

In some embodiments, an Fc variant is IgG2 Fc region. In some embodiments, an Fc variant comprises amino acid substitutions of A235E, D265G, A330S, and P331S. In some embodiments, an Fc variant is IgG2 Fc region and comprises amino acid substitutions of A235E, D265G, A330S, and P331S.

In one aspect, the present invention provides, among other things, an isolated polypeptide comprising an Fc variant of a wild-type human IgG Fc region, said Fc variant comprising an amino acid substitutions of 235E, D265G, and P329G, wherein the residues are numbered according to the EU index.

In some embodiments, an Fc variant is IgG2 Fc region. In some embodiments, an Fc variant comprises amino acid substitutions of A235E, D265G, and P329G. In some embodiments, an Fc variant is IgG2 Fc region and comprises amino acid substitutions of A235E, D265G, and P329G.

Design of Antibodies with Fc Variants

The Fc variants of the present invention may be an antibody, referred to herein as an “antibody of the present invention”. Antibodies of the present invention may comprise immunoglobulin sequences that are substantially encoded by immunoglobulin genes belonging to any of the antibody classes, including but not limited to IgG (including human subclasses IgG1, IgG2, IgG3, or IgG4), IgA (including human subclasses IgA1 and IgA2), IgD, IgE, IgG, and IgM classes of antibodies. Most preferably the antibodies of the present invention comprise sequences belonging to the human IgG class of antibodies. Antibodies of the present invention may be nonhuman, chimeric, humanized, or fully human. As will be appreciated by one skilled in the art, these different types of antibodies reflect the degree of “humanness” or potential level of immunogenicity in a human. For a description of these concepts, see Clark et al., 2000 and references cited therein (Clark, 2000, Immunol Today 21:397-402, incorporated by reference). Chimeric antibodies comprise the variable region of a nonhuman antibody, for example VH and VL domains of mouse or rat origin, operably linked to the constant region of a human antibody. Said nonhuman variable region may be derived from any organism as described above, preferably mammals and most preferably rodents or primates. In one embodiment, the antibody of the present invention comprises monkey variable domains, for example as described in Newman et al., 1992, Biotechnology 10:1455-1460, U.S. Pat. Nos. 5,658,570, and 5,750,105, incorporated by reference. In a preferred embodiment, the variable region is derived from a nonhuman source, but its immunogenicity has been reduced using protein engineering. In a preferred embodiment, the antibodies of the present invention are humanized (Tsurushita & Vasquez, 2004, Humanization of Monoclonal Antibodies, Molecular Biology of B Cells, 533-545, Elsevier Science (USA), incorporated by reference). By “humanized” antibody as used herein is meant an antibody comprising a human framework region (FR) and one or more complementarity determining regions (CDR's) from a non-human (usually mouse or rat) antibody. The non-human antibody providing the CDR's is called the “donor” and the human immunoglobulin providing the framework is called the “acceptor”. Humanization relies principally on the grafting of donor CDRs onto acceptor (human) VL and VH frameworks (Winter U.S. Pat. No. 5,225,539, incorporated by reference). This strategy is referred to as “CDR grafting”. “Backmutation” of selected acceptor framework residues to the corresponding donor residues is often required to regain affinity that is lost in the initial grafted construct (U.S. Pat. Nos. 5,530,101; 5,585,089; 5,693,761; 5,693,762; 6,180,370; 5,859,205; 5,821,337; 6,054,297; 6,407,213, incorporated by reference). A large number of other methods for humanization are known in the art (Tsurushita & Vasquez, 2004, Humanization of Monoclonal Antibodies, Molecular Biology of B Cells, 533-545, Elsevier Science (USA), incorporated by reference), and any of such methods may find use in the present invention for modifying Fc variants for reduced immunogenicity. The humanized antibody optimally also will comprise at least a portion of an immunoglobulin constant region, typically that of a human immunoglobulin, and thus will typically comprise a human Fc region.

In some embodiments, an antibody comprising an Fc variant is a monospecific antibody. In some embodiments, an antibody comprising an Fc variant is a multispecific antibody. In some embodiments, an antibody comprising an Fc variant is a bispecific antibody. In some embodiments, an antibody comprising an Fc variant is a multiparatopic antibody, e.g., comprises a plurality of immunoglobulin variable region sequences, wherein a first immunoglobulin variable region sequence of the plurality has binding specificity for a first epitope and a second immunoglobulin variable region sequence of the plurality has binding specificity for a second epitope. In an embodiment, the first and second epitopes are on the same antigen, e.g., the same protein (or subunit of a multimeric protein). A bispecific or biparatopic antibody has specificity for no more than two antigens or epitopes. A bispecific or biparatopic antibody molecule is typically characterized by a first immunoglobulin variable region sequence which has binding specificity for a first epitope and a second immunoglobulin variable region sequence that has binding specificity for a second epitope. In an embodiment, a bispecific or biparatopic antibody molecule comprises a half antibody, or fragment thereof, having binding specificity for a first epitope and a half antibody, or fragment thereof, having binding specificity for a second epitope. In an embodiment, the first and second epitopes are on the same antigen, e.g., the same protein (or subunit of a multimeric protein). In some embodiments, the biparatopic antibody presented in this disclosure comprises a Fab-Fc and a single chain variable fragment (scFv), wherein the Fc is linked to the scFv via a linker.

In some embodiments, the biparatopic antibody is a bispecific antibody with two arms single chain Fab-Fc design, comprising “knobs-in-holes” (KiH) mutations in CH3 domain, to assemble two half antibodies (common Fc heterodimer and unique VH—CH and VL—CL domains). In some embodiments, the KiH mutations comprise, a T366Y mutation in one CH3 domain can be used to create a knob while an Y407T mutation in the other CH3 domain to create a hole. In some embodiments, F405A mutation in one CH3 domain can be used to create a knob while a T394W mutation in the other CH3 domain to create a hole. In some embodiments, a T366W mutation in one CH3 domain can be used to create a knob while an Y407A mutation in the other CH3 domain to create a hole. In some embodiments, the biparatopic scFv-Fc molecules can be produced with knob-hole technology (e.g., including hole mutations: Y349C, T366S, L368A, Y407V; knob mutations: S354C, T366W).

In some embodiments, the biparatopic antibody comprises antibody formats described in Table 2.

TABLE 2
Formats of biparatopic antibodies

(Site II) Fab-IgG-scFv (Site I)
(Site II) Fab-IgG-linker-VL(Site I)-linker-VH (Site I)
(Site II) Fab-IgG-linker-VH(Site I)-linker-VL (Site I)
(Site I) Fab-IgG-scFv (Site II)

In an embodiment, the biparatopic antibody molecule comprises two heavy chain variable regions and two light chain variable regions. In an embodiment, the anti-C5aR1 antibody molecule comprises a Fab, F(ab′)2, Fv, Fd, or a single chain Fv fragment (scFv).

In some embodiments, the Fc domain used in this application comprises or is derived from an IgG, IgM, IgE, Fc portion. In addition to the KiH mutations described above, the Fc domain comprises S228P mutation. In some embodiments, the S228P enhanced the homogeneity of the antibody. In some embodiments, the Fc domain comprises or is derived from an IgG Fc domain. In some embodiments, the IgG Fc domain is IgG1, IgG2, IgG3 or IgG4 Fc domain. In some embodiments, the Fc domain is derived from or comprises an IgG4 Fc domain. In some embodiments, the Fc domain is derived from or comprises an IgG4 Fc domain with S228P mutation. In some embodiments, the Fc domain is derived from or comprises an IgG1 Fc domain. In some embodiments, the Fc domain is derived from or comprises an IgG1 Fc domain with S228P mutation.

In some exemplary embodiments, the mono-specific and biparatopic antibodies can be modified or mutated to enhance the thermal stability of the antibody. The thermal stability of the antibodies can be evaluated by determining the aggregation onset temperature. One of the ways to increase antibody stability is to raise the thermal transition midpoint (Tm) as measured by differential scanning calorimetry (DSC). In general, the protein Tm is correlated with its stability and inversely correlated with its susceptibility to unfolding and denaturation in solution and the degradation processes that depend on the tendency of the protein to unfold. A number of studies have found correlation between the ranking of the physical stability of formulations measured as thermal stability by DSC and physical stability measured by other methods (Maa et al. (1996) Int. J. Pharm. 140: 155-68; Remmele et al. (1997) Pharm. Res. 15: 200-8; Gupta et al. (2003) AAPS PharmSci. 5E8: 2003; Bedu-Addo et al. (2004) Pharm. Res. 21: 1353-61; Zhang et al. (2004) J. Pharm. Sci. 93: 3076-89). Formulation studies suggest that a Fab Tm has implication for long-term physical stability of a corresponding mAb.

In some exemplary embodiments, strategic introduction of disulfide bonds can stabilize monomeric and multisubunit proteins, play a role in enhancing thermal stability of antibodies.

In some exemplary embodiments, strategic introduction of it-stacking interactions with aromatic amino acids (AAs) like tryptophan (TRP), tyrosine (TYR), phenylalanine (PHE) and histidine (HIS), play a role in enhancing thermal stability of antibodies.

In some embodiments, strategic introduction of salt bridges occurring between amino acid side-chains with opposite positive or negative full-electron charges, namely, (at neutral pH) Glu or Asp vs. Arg or Lys, enhance the stability of proteins, particularly antibodies.

In some exemplary embodiments, the monospecific antibody or the biparatopic antibody comprise one or more thermal stability enhancing modifications. In some embodiments, the thermal stability enhancing modification is introduction of a cysteine residue.

In some embodiments, the Tm of exemplary biparatopic antibodies is greater than 65° C. In some embodiments, the Tm of exemplary biparatopic antibodies is greater than 60° C. the Tm of exemplary biparatopic antibodies is greater than 55° C. In some embodiments, the Tm of exemplary biparatopic antibodies is greater than 50° C.

In some embodiments, peptide linkers are used to link scFv or single chain antibodies to the Fc domain of the Fab. Several Examples of suitable linkers include a single glycine (G) residue; a diglycine peptide (GG); a tripeptide (GGG); a peptide with four glycine residues (GGGG); a peptide with five glycine residues (GGGGG); a peptide with six glycine residues (GGGGGG); a peptide with seven glycine residues (GGGGGGG); a peptide with eight glycine residues (GGGGGGGG). Other combinations of amino acid residues may be used such as the peptide GGGGS, the peptide GGGGSGGGGS, the peptide GGGGSGGGGSGGGGS, the peptide GGGGSGGGGSGGGGSGGGGS, the peptide GGSGSSGSGG, QRIEG and the peptide GQPKAAP. Other suitable linkers include a single Ser, and Val residue; the dipeptide RTQP, SS, TK, SL, TKGPS, TVAAP, QPKAA. The examples listed above are not intended to limit the scope of the disclosure in any way, and linkers comprising randomly selected amino acids selected from the group consisting of valine, leucine, isoleucine, serine, threonine, lysine, arginine, histidine, aspartate, glutamate, asparagine, glutamine, glycine, and proline have been shown to be suitable in the binding proteins. For additional descriptions of linker sequences, see, e.g., WO2012135345.

The identity and sequence of amino acid residues in the linker may vary depending on the type of secondary structural element necessary to achieve in the linker. For example, glycine, serine, and alanine are best for linkers having maximum flexibility. Some combination of glycine, proline, threonine, and serine are useful if a more rigid and extended linker is necessary. Any amino acid residue may be considered as a linker in combination with other amino acid residues to construct larger peptide linkers as necessary depending on the desired properties.

Targets

Virtually any antigen may be targeted by the Fc variants of the present invention, including but not limited to proteins, subunits, domains, motifs, and/or epitopes belonging to the following list of targets: 17-IA, 4-1 BB, 4Dc, 6-keto-PGF1a, 8-iso-PGF2a, 8-oxo-dG, Al Adenosine Receptor, A33, ACE, ACE-2, Activin, Activin A, Activin AB, Activin B, Activin C, Activin RIA, Activin RIA ALK-2, Activin RIB ALK-4, Activin RIIA, Activin RIM, ADAM, ADAM10, ADAM12, ADAM15, ADAM17/TACE, ADAMS, ADAMS, ADAMTS, ADAMTS4, ADAMTS5, Addressins, aFGF, ALCAM, ALK, ALK-1, ALK-7, alpha-1-antitrypsin, alpha-V/beta-1 antagonist, ANG, Ang, APAF-1, APE, APJ, APP, APRIL, AR, ARC, ART, Artemin, anti-Id, ASPARTIC, Atrial natriuretic factor, av/b3 integrin, Ax1, b2M, B7-1, B7-2, B7-H, B-lymphocyte Stimulator (BlyS), BACE, BACE-1, Bad, BAFF, BAFF-R, Bag-1, BAK, Bax, BCA-1, BCAM, Bcl, BCMA, BDNF, b-ECGF, bFGF, BID, Bik, BIM, BLC, BL-CAM, BLK, BMP, BMP-2 BMP-2a, BMP-3 Osteogenin, BMP-4 BMP-2b, BMP-5, BMP-6 Vgr-1, BMP-7 (0P-1), BMP-8 (BMP-8a, OP-2), BMPR, BMPR-IA (ALK-3), BMPR-IB (ALK-6), BRK-2, RPK-1, BMPR-II (BRK-3), BMPs, b-NGF, BOK, Bombesin, Bone-derived neurotrophic factor, BPDE, BPDE-DNA, BTC, complement factor 3 (C3), C3a, C3b, C4, C5, C5a, C5a Receptor 1 (C5aR1) C10, CA125, CAD-8, Calcitonin, cAMP, carcinoembryonic antigen (CEA), carcinoma-associated antigen, Cathepsin A, Cathepsin B, Cathepsin C/DPPI, Cathepsin D, Cathepsin E, Cathepsin H, Cathepsin L, Cathepsin O, Cathepsin S, Cathepsin V, Cathepsin X/Z/P, CBL, CCI, CCK2, CCL, CCL1, CCL11, CCL12, CCL13, CCL14, CCL15, CCL16, CCL17, CCL18, CCL19, CCL2, CCL20, CCL21, CCL22, CCL23, CCL24, CCL25, CCL26, CCL27, CCL28, CCL3, CCL4, CCL5, CCL6, CCL7, CCL8, CCL9/10, CCR, CCR1, CCR10, CCR10, CCR2, CCR3, CCR4, CCR5, CCR6, CCR7, CCR8, CCR9, CD1, CD2, CD3, CD3E, CD4, CD5, CD6, CD7, CD8, CD10, CD11a, CD11b, CD11c, CD13, CD14, CD15, CD16, CD18, CD19, CD20, CD21, CD22, CD23, CD25, CD27L, CD28, CD29, CD30, CD3OL, CD32, CD33 (p67 proteins), CD34, CD38, CD40, CD40L, CD44, CD45, CD46, CD49a, CD52, CD54, CD55, CD56, CD61, CD64, CD66e, CD74, CD80 (B7-1), CD89, CD95, CD123, CD137, CD138, CD140a, CD146, CD147, CD148, CD152, CD164, CEACAM5, CFTR, cGMP, CINC, Clostridium botulinum toxin, Clostridium perfringens toxin, CKb8-1, CLC, CMV, CMV UL, CNTF, CNTN-1, COX, C-Ret, CRG-2, CT-1, CTACK, CTGF, CTLA-4, CX3CL1, CX3CR1, CXCL, CXCL1, CXCL2, CXCL3, CXCL4, CXCL5, CXCL6, CXCL7, CXCL8, CXCL9, CXCL10, CXCL11, CXCL12, CXCL13, CXCL14, CXCL15, CXCL16, CXCR, CXCR1, CXCR2, CXCR3, CXCR4, CXCR5, CXCR6, cytokeratin tumor-associated antigen, DAN, DCC, DcR3, DC-SIGN, Decay accelerating factor, des(1-3)-IGF-1 (brain IGF-1), Dhh, digoxin, DNAM-1, Dnase, Dpp, DPPIV/CD26, Dtk, ECAD, EDA, EDA-Al, EDA-A2, EDAR, EGF, EGFR (ErbB-1), EMA, EMMPRIN, ENA, endothelin receptor, Enkephalinase, eNOS, Eot, eotaxin1, EpCAM, Ephrin B2/EphB4, EPO, ERCC, E-selectin, ET-1, Factor IIa, Factor VII, Factor VIIIc, Factor IX, fibroblast activation protein (FAP), Fas, FcR1, FEN-1, Ferritin, FGF, FGF-19, FGF-2, FGF3, FGF-8, FGFR, FGFR-3, Fibrin, FL, FLIP, Flt-3, Flt-4, Follicle stimulating hormone, Fractalkine, FZD1, FZD2, FZD3, FZD4, FZD5, FZD6, FZD7, FZD8, FZD9, FZD10, G250, Gas 6, GCP-2, GCSF, GD2, GD3, GDF, GDF-1, GDF-3 (Vgr-2), GDF-5 (BMP-14, CDMP-1), GDF-6 (BMP-13, CDMP-2), GDF-7 (BMP-12, CDMP-3), GDF-8 (Myostatin), GDF-9, GDF-15 (MIC-1), GDNF, GDNF, GFAP, GFRa-1, GFR-alpha1, GFR-alpha2, GFR-alpha3, GITR, Glucagon, Glut 4, glycoprotein IIb/IIIa (GPI1b/111a), GM-CSF, gp130, gp72, GRO, Growth hormone releasing factor, Hapten (NP-cap or NIP-cap), HB-EGF, HCC, HCMV gB envelope glycoprotein, HCMV) gH envelope glycoprotein, HCMV UL, Hemopoietic growth factor (HGF), Hep B gp120, heparanase, Her2, Her2/neu (ErbB-2), Her3 (ErbB-3), Her4 (ErbB-4), herpes simplex virus (HSV) gB glycoprotein, HSV gD glycoprotein, HGFA, High molecular weight melanoma-associated antigen (HMW-MAA), HIV gp120, HIV IIIB gp120 V3 loop, HLA, HLA-DR, HM1.24, HMFG PEM, HRG, Hrk, human cardiac myosin, human cytomegalovirus (HCMV), human growth hormone (HGH), HVEM, 1-309, IAP, ICAM, ICAM-1, ICAM-3, ICE, ICOS, IFNg, Ig, IgA receptor, IgE, IGF, IGF binding proteins, IGF-1R, IGFBP, IGF-I, IGF-II, IL, IL-1, IL-1R, IL-2, IL-2R, IL-4, IL-4R, IL-5, IL-5R, IL-6, IL-6R, IL-8, IL-9, IL-10, IL-12, IL-13, IL-15, IL-18, IL-18R, IL-23, interferon (INF)-alpha, INF-beta, INF-gamma, Inhibin, iNOS, Insulin A-chain, Insulin B-chain, Insulin-like growth factor 1, integrin alpha2, integrin alpha3, integrin alpha4, integrin alpha4/beta1, integrin alpha4/beta7, integrin alpha5 (alphaV), integrin alpha5/beta1, integrin alpha5/beta3, integrin alpha6, integrin beta1, integrin beta2, interferon gamma, IP-10, I-TAC, JE, Kallikrein 2, Kallikrein 5, Kallikrein 6, Kallikrein 11, Kallikrein 12, Kallikrein 14, Kallikrein 15, Kallikrein L1, Kallikrein L2, Kallikrein L3, Kallikrein L4, KC, KDR, Keratinocyte Growth Factor (KGF), laminin 5, LAMP, LAP, LAP (TGF-1), Latent TGF-1, Latent TGF-1 bpi, LBP, LDGF, LECT2, Lefty, Lewis-Y antigen, Lewis-Y related antigen, LFA-1, LFA-3, Lfo, LIF, LIGHT, lipoproteins, LIX, LKN, Lptn, L-Selectin, LT-a, LT-b, LTB4, LTBP-1, Lung surfactant, Luteinizing hormone, Lymphotoxin Beta Receptor, Mac-1, MAdCAM, MAG, MAP2, MARC, MCAM, MCAM, MCK-2, MCP, M-CSF, MDC, Mer, METALLOPROTEASES, MGDF receptor, MGMT, MHC (HLA-DR), MIF, MIG, MW, MIP-1-alpha, MK, MMAC1, MMP, MMP-1, MMP-10, MMP-11, MMP-12, MMP-13, MMP-14, MMP-15, MMP-2, MMP-24, MMP-3, MMP-7, MMP-8, MMP-9, MPIF, Mpo, MSK, MSP, mucin (Mud), MUC18, Muellerian-inhibitin substance, Mug, MuSK, NAIP, NAP, NCAD, N-Cadherin, NCA 90, NCAM, NCAM, Neprilysin, Neurotrophin-3, -4, or -6, Neurturin, Neuronal growth factor (NGF), NGFR, NGF-beta, nNOS, NO, NOS, Npn, NRG-3, NT, NTN, OB, OGG1, OPG, OPN, OSM, OX40L, OX4OR, p150, p95, PADPr, Parathyroid hormone, PARC, PARP, PBR, PBSF, PCAD, P-Cadherin, PCNA, PDGF, PDGF, PDK-1, PECAM, PEM, PF4, PGE, PGF, PGI2, PGJ2, PIN, PLA2, placental alkaline phosphatase (PLAP), PIGF, PLP, PP14, Proinsulin, Prorelaxin, Protein C, PS, PSA, PSCA, prostate specific membrane antigen (PSMA), PTEN, PTHrp, Ptk, PTN, R51, RANK, RANKL, RANTES, RANTES, Relaxin A-chain, Relaxin B-chain, renin, respiratory syncytial virus (RSV) F, RSV Fgp, Ret, Rheumatoid factors, RLIP76, RPA2, RSK, 5100, SCF/KL, SDF-1, SERINE, Serum albumin, sFRP-3, Shh, SIGIRR, SK-1, SLAM, SLPI, SMAC, SMDF, SMOH, SOD, SPARC, Stat, STEAP, STEAP-II, TACE, TACI, TAG-72 (tumor-associated glycoprotein-72), TARC, TCA-3, T-cell receptors (e.g., T-cell receptor alpha/beta), TdT, TECK, TEM1, TEM5, TEM7, TEM8, TERT, testicular PLAP-like alkaline phosphatase, TfR, TGF, TGF-alpha, TGF-beta, TGF-beta Pan Specific, TGF-beta R1 (ALK-5), TGF-beta RII, TGF-beta RIIb, TGF-beta RIII, TGF-beta1, TGF-beta2, TGF-beta3, TGF-beta4, TGF-beta5, Thrombin, Thymus Ck-1, Thyroid stimulating hormone, Tie, TIMP, TIQ, Tissue Factor, TMEFF2, Tmpo, TMPRSS2, TNF, TNF-alpha, TNF-alpha beta, TNF-beta2, TNFα, TNF-R1, TNF-RII, TNFRSF10A (TRAIL R1Apo-2, DR4), TNFRSF10B (TRAIL R2DR5, KILLER, TRICK-2A, TRICK-B), TNFRSF10C (TRAIL R3DcR1, LIT, TRID), TNFRSF10D (TRAIL R4 DcR2, TRUNDD), TNFRSF11A (RANK ODF R, TRANCE R), TNFRSF11B (OPG OCIF, TR1), TNFRSF12 (TWEAK R FN14), TNFRSF13B (TACI), TNFRSF13C (BAFF R), TNFRSF14 (HVEM ATAR, HveA, LIGHT R, TR2), TNFRSF16 (NGFR p75NTR), TNFRSF17 (BCMA), TNFRSF18 (GITR AITR), TNFRSF19 (TROY TAJ, TRADE), TNFRSF19L (RELT), TNFRSF1A (TNF R1CD120a, p55-60), TNFRSF1B (TNF RIICD120b, p′75-80), TNFRSF26 (TNFRH3), TNFRSF3 (LTbR TNF RIII, TNFC R), TNFRSF4 (OX40 ACT35, TXGP1R), TNFRSF5 (CD40 p50), TNFRSF6 (Fas Apo-1, APT1, CD95), TNFRSF6B (DcR3M68, TR6), TNFRSF7 (CD27), TNFRSF8 (CD30), TNFRSF9 (4-1BB CD137, ILA), TNFRSF21 (DR6), TNFRSF22 (DcTRAIL R2TNFRH2), TNFRST23 (DcTRAIL R1TNFRH1), TNFRSF25 (DR3 Apo-3, LARD, TR-3, TRAMP, WSL-1), TNFSF10 (TRAIL Apo-2 Ligand, TL2), TNFSF11 (TRANCE/RANK Ligand ODF, OPG Ligand), TNFSF12 (TWEAK Apo-3 Ligand, DR3 Ligand), TNFSF13 (APRIL TALL2), TNFSF13B (BAFF BLYS, TALL1, THANK, TNFSF20), TNFSF14 (LIGHT HVEM Ligand, LTg), TNFSF15 (TL1A/VEGI), TNFSF18 (GITR Ligand AITR Ligand, TL6), TNFSF1A (TNF-a Conectin, DIF, TNFSF2), TNFSF1B (TNF-b LTa, TNFSF1), TNFSF3 (LTb TNFC, p33), TNFSF4 (OX40 Ligand gp34, TXGP1), TNFSF5 (CD40 Ligand CD154, gp39, HIGM1, IMD3, TRAP), TNFSF6 (Fas Ligand Apo-1 Ligand, APT1 Ligand), TNFSF7 (CD27 Ligand CD70), TNFSF8 (CD30 Ligand CD153), TNFSF9 (4-1BB Ligand CD137 Ligand), TP-1, t-PA, Tpo, TRAIL, TRAIL R, TRAIL-R1, TRAIL-R2, TRANCE, transferring receptor, TRF, Trk, TROP-2, TSG, TSLP, tumor-associated antigen CA 125, tumor-associated antigen expressing Lewis Y related carbohydrate, TWEAK, TXB2, Ung, uPAR, uPAR-1, Urokinase, VCAM, VCAM-1, VECAD, VE-Cadherin, VE-cadherin-2, VEFGR-1 (flt-1), VEGF, VEGFR, VEGFR-3 (flt-4), VEGI, VIM, Viral antigens, VLA, VLA-1, VLA-4, VNR integrin, von Willebrands factor, WIF-1, WNT1, WNT2, WNT2B/13, WNT3, WNT3A, WNT4, WNT5A, WNT5B, WNT6, WNT7A, WNT7B, WNT8A, WNT8B, WNT9A, WNT9A, WNT9B, WNT10A, WNT10B, WNT11, WNT16, XCL1, XCL2, XCR1, XCR1, XEDAR, XIAP, XPD, and receptors for hormones and growth factors.

In some embodiments, an antibody comprising an Fc variant specifically binds a complement factor. In some embodiments, an antibody comprising an Fc variant specifically binds SARS-CoV-2. In some embodiments, an antibody comprising an Fc variant specifically binds amyloid beta protofibrils. In some embodiments, an antibody comprising an Fc variant specifically binds LAG-3. In some embodiments, an antibody comprising an Fc variant specifically binds CD3. In some embodiments, an antibody comprising an Fc variant specifically binds VEGF. In some embodiments, an antibody comprising an Fc variant specifically binds Ang-2. In some embodiments, an antibody comprising an Fc variant specifically binds PD-1. In some embodiments, an antibody comprising an Fc variant specifically binds EGFR. In some embodiments, an antibody comprising an Fc variant specifically binds IFNAR1. In some embodiments, an antibody comprising an Fc variant specifically binds CD19. In some embodiments, an antibody comprising an Fc variant specifically binds IL-17A. In some embodiments, an antibody comprising an Fc variant specifically binds IL-17B. In some embodiments, an antibody comprising an Fc variant specifically binds IL-13. In some embodiments, an antibody comprising an Fc variant specifically binds angiopoietin-like 3. In some embodiments, an antibody comprising an Fc variant specifically binds nerve growth factor. In some embodiments, an antibody comprising an Fc variant specifically binds Ebola virus. In some embodiments, an antibody comprising an Fc variant specifically binds HER2. In some embodiments, an antibody comprising an Fc variant specifically binds GD2. In some embodiments, an antibody comprising an Fc variant specifically binds BCMA. In some embodiments, an antibody comprising an Fc variant specifically binds IL-6R. In some embodiments, an antibody comprising an Fc variant specifically binds TROP-2. In some embodiments, an antibody comprising an Fc variant specifically binds IGF-1R. In some embodiments, an antibody comprising an Fc variant specifically binds CD38. In some embodiments, an antibody comprising an Fc variant specifically binds Nectin-4. In some embodiments, an antibody comprising an Fc variant specifically binds P-selectin. In some embodiments, an antibody comprising an Fc variant specifically binds CD79b. In some embodiments, an antibody comprising an Fc variant specifically binds sclerostin. In some embodiments, an antibody comprising an Fc variant specifically binds IFNgamma. In some embodiments, an antibody comprising an Fc variant specifically binds CCR4. In some embodiments, an antibody comprising an Fc variant specifically binds CGRP receptor. In some embodiments, an antibody comprising an Fc variant specifically binds a receptor for complement factor. In some embodiments, an antibody comprising an Fc variant specifically binds C5a receptor 1. In some embodiments, an antibody comprising an Fc variant specifically binds C5a. In some embodiments, an antibody comprising an Fc variant specifically binds C3. In some embodiments, an antibody comprising an Fc variant specifically binds C3a. In some embodiments, an antibody comprising an Fc variant specifically binds C3b. In some embodiments, an antibody comprising an Fc variant specifically binds C3 receptor. In some embodiments, an antibody comprising an Fc variant specifically binds C10.

Vectors

Further provided herein are vectors that comprise nucleotide sequences encoding an isolated polypeptide comprising an Fc variant, as described herein.

In an embodiment, the vector comprises a nucleic acid described herein. For example, the vector can comprises a first and second nucleic acid encoding heavy and light chain variable regions, respectively, of an antibody molecule chosen from one or more of the antibody molecules disclosed herein.

In certain embodiments, the vector comprises a nucleotide sequence encoding an Fc variant region, or a sequence substantially homologous thereto (e.g., a sequence at least about 85%, 90%, 95%, 99% or more identical thereto, and/or having one or more substitutions, e.g., conserved substitutions).

The vectors include, but are not limited to, a virus, plasmid, cosmid, lambda phage or a yeast artificial chromosome (YAC). Numerous vector systems can be employed. For example, one class of vectors utilizes DNA elements which are derived from animal viruses such as, for example, bovine papilloma virus, polyoma virus, adenovirus, vaccinia virus, baculovirus, retroviruses (Rous Sarcoma Virus, MMTV or MOMLV) or SV40 virus. Another class of vectors utilizes RNA elements derived from RNA viruses such as Semliki Forest virus, Eastern Equine Encephalitis virus and Flaviviruses.

Additionally, cells which have stably integrated the DNA into their chromosomes may be selected by introducing one or more markers which allow for the selection of transfected host cells. The marker may provide, for example, prototropy to an auxotrophic host, biocide resistance (e.g., antibiotics), or resistance to heavy metals such as copper, or the like. The selectable marker gene can be either directly linked to the DNA sequences to be expressed or introduced into the same cell by cotransformation. Additional elements may also be needed for optimal synthesis of mRNA. These elements may include splice signals, as well as transcriptional promoters, enhancers, and termination signals.

Once the expression vector or DNA sequence containing the constructs has been prepared for expression, the expression vectors may be transfected or introduced into an appropriate host cell. Various techniques may be employed to achieve this, such as, for example, protoplast fusion, calcium phosphate precipitation, electroporation, retroviral transduction, viral transfection, gene gun, lipid-based transfection or other conventional techniques. In the case of protoplast fusion, the cells are grown in media and screened for the appropriate activity.

Methods and conditions for culturing the resulting transfected cells and for recovering the antibody molecule produced are known to those skilled in the art and may be varied or optimized depending upon the specific expression vector and mammalian host cell employed, based upon the present description.

Therapeutic Use

An isolated polypeptide comprising a Fc variant (e.g., an antibody) of the present invention can be used for treatment of various diseases including but not limited to: Melanoma, wAMD, DME, Esophageal squamous cell carcinoma, Type 1 diabetes, non-small lung cancer, asthma, CNS, cervical cancer, cold agglutinin disease, systemic lupus erythematosus, psoriasis, atopic dermatitis, endometrial cancer, bladder cancer, Ebola infection, HER2+breast cancer, multiple myeloma, breast cancer, thyroid eye disease, sickle cell disease, HIV infection, gastric cancer, anthrax infection, bone loss, Crohn disease, ANCA-associated vasculitis, lupus, rheumatoid arthritis, inflammatory bowel disease, C3 glomerulopathy (C3G), C3 Glomerulonephritis (C3GN), Dense Deposit Disease (DDD), hidradenitis suppurativa (HS), atypical hemolytic uremic syndrome, Lupus nephritis, IgA nephropathy, mayasthenia gravis, macular degeneration, Alzheimers Disease, Amylotrophic Lateral Sclerosis, Huntington's Disease, neuropathic pain, COVID-19 infection, allergic asthma, chronic obstructive pulmonary disease, bullous pemphigoid, pyoderma gangrenosum, psoriasis, Paroxysmal Nocturnal Hemoglobinuria with extra vascular hemolysis, acute kidney injury (AKI), chronic kidney disease (CKD), Geographic atrophy (GA), autoimmune hemolytic anemia (AIHA), and Age-related macular degeneration (AMD).

In some embodiments, the present invention provides a method of treating a disease or disorder, said method comprising administering a therapeutically effective amount of an isolated polypeptide comprising an Fc variant to a subject in need of. In some embodiments, the disease or disorder is ANCA-associated vasculitis. In some embodiments, the disease or disorder is C3 glomerulopathy (C3G).

In some embodiments, an Fc variant of the present invention can be engineered into an antibody that specifically binds to an antigen. In some embodiments, an Fc variant of the present invention can be engineered into an antibody listed in Table 3.

TABLE 3
Commercially available Therapeutic Antibody

| Name | Target | Specificity | Isotype |
|---|---|---|---|
| Muromonab-CD3 | CD3 | Monospecific | mIgG2a |
| Efalizumab | CD11a | Monospecific | hIgG1 |
| Tositumomab-I131 | CD20 | Monospecific | mIgG2a |
| Nebacumab | Endotoxin | Monospecific | hIgM |
| Edrecolomab | EpCAM | Monospecific | mIgG2a |
| Catumaxomab | EPCAM, CD3 | Bispecific | mIgG2a/k and |
| | | | rIgG2b/λ Hybrid |
| Daclizumab | CD25 | Monospecific | hIgG1 |
| Abciximab | GPIIb/IIIa | Monospecific | hIgG1 Fab |
| Rituximab | CD20 | Monospecific | hIgG1 |
| Basiliximab | IL-2R | Monospecific | hIgG1 |
| Palivizumab | RSV | Monospecific | hIgG1 |
| Infliximab | TNF | Monospecific | hIgG1 |
| Trastuzumab | HER2 | Monospecific | hIgG1 |
| Adalimumab | TNF | Monospecific | hIgG1 |
| Ibritumomab tiuxetan | CD20 | Monospecific | mIgG1 |
| Omalizumab | IgE | Monospecific | hIgG1 |
| Cetuximab | EGFR | Monospecific | hIgG1 |
| Bevacizumab | VEGF | Monospecific | hIgG1 |
| Natalizumab | a4 integrin | Monospecific | hIgG4 |
| Panitumumab | EGFR | Monospecific | hIgG2 |
| Ranibizumab | VEGF | Monospecific | hIgG1 Fab |
| Eculizumab | C5 | Monospecific | hIgG2(CH1-hinge)/ |
| | | | hIgG4(CH2-CH3) |
| Certolizumab pegol | TNF | Monospecific | hFab |
| Ustekinumab | IL-12/23 | Monospecific | hIgG1 |
| Canakinumab | IL-1β | Monospecific | hIgG1 |
| Golimumab | TNF | Monospecific | hIgG1 |
| Ofatumumab | CD20 | Monospecific | hIgG1 |
| Tocilizumab | IL-6R | Monospecific | hIgG1 |
| Denosumab | RANK-L | Monospecific | hIgG2 |
| Belimumab | BLyS | Monospecific | hIgG1 |
| Ipilimumab | CTLA-4 | Monospecific | hIgG1 |
| Brentuximab vedotin | CD30 | Monospecific | hIgG1 |
| Pertuzumab | HER2 | Monospecific | hIgG1 |
| Ado-trastuzumab emtansine | HER2 | Monospecific | hIgG1 |
| Raxibacumab | B. anthrasis PA | Monospecific | hIgG1 |
| Obinutuzumab | CD20 | Monospecific | hIgG1 |
| Siltuximab | IL-6 | Monospecific | hIgG1 |
| Ramucirumab | VEGFR2 | Monospecific | hIgG1 |
| Vedolizumab | α4β7 integrin | Monospecific | hIgG1 |
| Nivolumab | PD1 | Monospecific | hIgG4 |
| Pembrolizumab | PD1 | Monospecific | hIgG4 |
| Blinatumomab | CD19, CD3 | Bispecific | Tandem scFv |
| Alemtuzumab | CD52 | Monospecific | hIgG1 |
| Evolocumab | PCSK9 | Monospecific | hIgG2 |
| Idarucizumab | Dabigatran | Monospecific | hIgG1 Fab |
| Necitumumab | EGFR | Monospecific | hIgG1 |
| Dinutuximab | GD2 | Monospecific | hIgG1 |
| Secukinumab | IL-17a | Monospecific | hIgG1 |
| Mepolizumab | IL-5 | Monospecific | hIgG1 |
| Alirocumab | PCSK9 | Monospecific | hIgG1 |
| Daratumumab | CD38 | Monospecific | hIgG1 |
| Elotuzumab | SLAMF7 | Monospecific | hIgG1 |
| Ixekizumab | IL-17a | Monospecific | hIgG4 |
| Reslizumab | IL-5 | Monospecific | hIgG4 |
| Olaratumab | PDGFRα | Monospecific | hIgG1 |
| Bezlotoxumab | Clostridium difficile | Monospecific | hIgG1 |
| | enterotoxin B | | |
| Atezolizumab | PD-L1 | Monospecific | hIgG1 |
| Obiltoxaximab | B. anthrasis PA | Monospecific | hIgG1 |
| Brodalumab | IL-17R | Monospecific | hIgG2 |
| Dupilumab | IL-4R α | Monospecific | hIgG4 |
| Inotuzumab ozogamicin | CD22 | Monospecific | hIgG4 |
| Guselkumab | IL-23 p19 | Monospecific | hIgG1 |
| Sarilumab | IL-6R | Monospecific | hIgG1 |
| Avelumab | PD-L1 | Monospecific | hIgG1 |
| Emicizumab | Factor Ixa, X | Bispecific | hIgG4 |
| Ocrelizumab | CD20 | Monospecific | hIgG1 |
| Benralizumab | IL-5R α | Monospecific | hIgG1 |
| Durvalumab | PD-L1 | Monospecific | hIgG1 |
| Gemtuzumab ozogamicin | CD33 | Monospecific | hIgG4 |
| Erenumab (erenumab-aooe) | CGRP receptor | Monospecific | hIgG2 |
| Galcanezumab | CGRP | Monospecific | hIgG4 |
| (galcanezumab-gnlm) | | | |
| Burosumab | FGF23 | Monospecific | hIgG1 |
| (burosumab-twza) | | | |
| Lanadelumab | Plasma kallikrelin | Monospecific | hIgG1 |
| (lanadelumab-flyo) | | | |
| Mogamulizumab | CCR4 | Monospecific | hIgG1 |
| (mogamulizumab-kpkc) | | | |
| Tildrakizumab | IL-23 p19 | Monospecific | hIgG1 |
| (tildrakizumab-asmn) | | | |
| Fremanezumab | CGRP | Monospecific | hIgG2Δa |
| (fremanezumab-vfrm) | | | |
| Ravulizumab | C5 | Monospecific | hIgG2(CH1-hinge)/ |
| (ravulizumab-cwvz) | | | hIgG4(CH2-CH3) |
| Cemiplimab | PD-1 | Monospecific | hIgG4 |
| (cemiplimab-rwlc) | | | |
| Ibalizumab | CD4 | Monospecific | hIgG4 |
| (ibalizumab-uiyk) | | | |
| Emapalumab | IFNg | Monospecific | hIgG1 |
| (emapalumab-lzsg) | | | |
| Moxetumomab pasudotox | CD22 | Monospecific | mIgG1 dsFv fused |
| (moxetumomab pasudotox-tdfk) | | | with PE38 exotoxin |
| Caplacizumab | von Willebrand factor | Monospecific | bivalent Nanobody |
| (caplacizumab-yhdp) | | | |
| Risankizumab | IL-23 p19 | Monospecific | hIgG1 |
| (risankizumab-rzaa) | | | |
| Polatuzumab vedotin | CD79b | Monospecific | hIgG1 |
| (polatuzumab vedotin-piiq) | | | |
| Romosozumab | Sclerostin | Monospecific | hIgG2 |
| (romosozumab-aqqg) | | | |
| Brolucizumab | VEGF-A | Monospecific | hscFv |
| (brolucizumab-dbll) | | | |
| Crizanlizumab | CD62 (aka P-selectin) | Monospecific | hIgG2 |
| (crizanlizumab-tmca) | | | |
| Enfortumab vedotin | Nectin-4 | Monospecific | hIgG1 |
| (enfortumab vedotin-ejfv) | | | |
| [fam-]trastuzumab | HER2 | Monospecific | hIgG1 |
| deruxtecan, (fam- | | | |
| trastuzumab deruxtecan-nxki) | | | |
| Teprotumumab | IGF-1R | Monospecific | hIgG1 |
| (teprotumumab-trbw) | | | |
| Eptinezumab | CGRP | Monospecific | hIgG1 |
| (eptinezumab-jjmr) | | | |
| Isatuximab | CD38 | Monospecific | hIgG1 |
| (isatuximab-irfc) | | | |
| Sacituzumab govitecan | TROP-2 | Monospecific | hIgG1 |
| (Sacituzumab govitecan-hziy) | | | |
| Inebilizumab | CD19 | Monospecific | hIgG1 |
| (inebilizumab-cdon) | | | |
| Tafasitamab | CD19 | Monospecific | hIgG1/2 hybrid |
| (tafasitamab-cxix) | | | |
| Belantamab mafodotin | B-cell maturation | Monospecific | hIgG1 |
| (belantamab | antigen | | |
| mafodotin-blmf) | | | |
| Satralizumab | IL-6R | Monospecific | hIgG2 |
| (satralizumab-mwge) | | | |
| Atoltivimab, maftivimab, | Ebola virus | Monospecific | hIgG1 |
| and odesivimab-ebgn | | | |
| Naxitamab-gqgk | GD2 | Monospecific | hIgG1 |
| Margetuximab-cmkb | HER2 | Monospecific | hIgG1 |
| Ansuvimab-zykl | Ebola virus | Monospecific | hIgG1 |
| | glycoprotein | | |
| Evinacumab | Angiopoietin-like 3 | Monospecific | hIgG4 |
| Loncastuximab tesirine | CD19 | Monospecific | hIgG1/k |
| Dostarlimab | PD-1 | Monospecific | hIgG4 |
| Amivantamab | EGFR, cMET | Bispecific | hIgG1 |
| Aducanumab | Amyloid beta | Monospecific | hIgG1 |
| Tanezumab | Nerve growth factor | Monospecific | hIgG2Δa |
| Tralokinumab | IL-13 | Monospecific | hIgG4 |
| Teplizumab | CD3 | Monospecific | hIgG1 |
| Narsoplimab | MASP-2 | Monospecific | hIgG4 |
| Retifanlimab | PD-1 | Monospecific | hIgG4 |
| Oportuzumab monatox | EpCAM | Monospecific | hscFv fused with |
| | | | PE38 exotoxin |
| Anifrolumab, | IFNAR1 | Monospecific | hIgG1 |
| anifrolumab-fnia | | | |
| Inolimomb | CD25 | Monospecific | mIgG1 |
| Bimekizumab | IL-17A and IL-17F | Monospecific | hIgG1 |
| | (overlapping binding | | |
| | site) | | |
| Sutimlimab | C1s | Monospecific | hIgG4 |
| Ublituximab | CD20 | Monospecific | hIgG1 |
| Tisotumab vedotin, | Tissue factor | Monospecific | hIgG1 |
| tisotumab vedotin-tftv | | | |
| Toripalimab | PD-1 | Monospecific | hIgG4 |
| Sintilimab | PD-1 | Monospecific | hIgG4 |
| Tezepelumab | Thymic stromal | Monospecific | hIgG2 |
| | lymphopoietin | | |
| Omburtamab | B7-H3 (CD276) | Monospecific | hIgG1 |
| Penpulimab | PD-1 | Monospecific | hIgG1 |
| Faricimab | VEGF-A, Ang-2 | Bispecific | hIgG1 |
| Tebentafusp | gp100, CD3 | Bispecific | hscFV fused |
| | | | with a TCR |
| Tislelizumab | PD-1 | Monospecific | hIgG4 |
| Relatlimab | LAG-3 | Monospecific | hIgG4 |
| Lecanemab | Amyloid beta | Monospecific | hIgG1 |
| | protofibrils | | |
| Casirivimab + imdevimab | SARS-CoV-2 | Monospecific | hIgG1 |
| Regdanvimab | SARS-CoV-2 | Monospecific | hIgG1 |
| Nimotuzumab | EGFR | Monospecific | hIgG1 |
| Itolizumab | CD6 | Monospecific | hIgG1 |
| Rmab | rabies virus G | Monospecific | hIgG1 |
| | glycoprotein | | |
| Sotrovimab | SARS-CoV-2 | Monospecific | |
| Regdanvimab | SARS-CoV-2 | Monospecific | |

### EXAMPLES

Other features, objects, and advantages of the present disclosure are apparent in the examples that follow. It should be understood, however, that the examples, while indicating embodiments of the present disclosure, are given by way of illustration only, not limitation. Various changes and modifications within the scope of the disclosure will become apparent to those skilled in the art from the examples.

### Example 1

### Generations of New Fc Mutations

Fc-Gamma receptors recognize and bind the Fc region of IgG antibodies. This binding modulates immune response by triggering effector functions. In some disease indications it may be beneficial for a therapeutic antibody to interact with Fc-Gamma receptors and enhance native Fc-Gamma receptor activation, but for other indications it can be detrimental. In cases where additional activation is detrimental, Fc-engineering is required to silence the IgG Fc domain such that it cannot bind to Fc-Gamma receptors. In this example, new Fc variants were engineered, that are particularly effective in silencing the IgG Fc domain. Notably, the combinations of the Fc mutations of the present invention are new and are effective in abolishing the affinity for Fc-Gamma receptors and C1q. The Fc variants of the present invention are shown in Table 1.

TABLE 1
Novel Fc Variants

| Fc | Mutations | Subtype |
|---|---|---|
| VFc07 | L234F/L235E/D265G | IgG1 |
| VFc08 | L234F/L235E/G237A/D265G | IgG1 |
| VFc09 | L234V/L235E/G237A/D265G | IgG1 |
| VFc10 | L234F/L235E/D265G/A330S/P331S | IgG1 |
| VFc11 | L234V/L235A/G237A/D265G | IgG1 |
| VFc12 | L234V/L235A/G237A/D265G/A330S/P331S | IgG1 |
| VFc13 | D265G | IgG1 |
| VFc16 | L235E/D265G | IgG4-S228P |
| VFc17 | F234V/L235E/D265G | IgG4-S228P |
| VFc18 | F234V/L235A/G237A/D265G | IgG4-S228P |
| VFc19 | L235E/G237A/D265G | IgG4-S228P |
| VFc20 | L235E/G237A/P329G | IgG4-S228P |
| VFc21 | L235E/G237A/L328R | IgG4-S228P |
| VFc22 | D265G/A330S/P331S | IgG2 |
| VFc23 | A235E/D265G/A330S/P331S | IgG2 |
| VFc24 | A235E/D265G/P329G | IgG2 |

### Example 2

### Fc Variants Displayed Significantly Reduced Fcγ Receptor and C1q Binding

This example confirms that the new Fc variants shown in Table 1 successfully abolished Fc-Gamma receptor and C1q binding. The Fc variants were engineered into an antibody “Ab1” and “Ab2”, each of which comprises a distinct variable domain and targets a different antigen.

Method of Determining Fc-Gamma Receptor and C1q Binding

This protocol describes methods which can be used to determine the extent to which Fc-silenced antibodies bind to Fc-Gamma receptors or C1q using biolayer interferometry (BLI) on the Octet Red 384 system or ELISA. FcγRI, FcγRIIa H167, FcγRIIb, FcγRIIIa V176 and FcγRIIIb bindings were evaluated using either NiNTA of HIS1K biosensors. In both procedures, Fc-Gamma receptors were first loaded onto biosensors, which were then blocked with either casein or BSA to prevent non-specific binding of the antibodies to any unbound ligand on the biosensor. Next, the biosensors were introduced to antibody before being returned to blocking buffer for a brief dissociation phase. The resulting binding sensograms were used to qualitatively evaluate the strength of each measured interaction while the maximum equilibrium binding response were used as a quantitative readout, with maximum equilibrium binding response being proportional to binding affinity. Binding to C1q was measured in a similar manner, according to known methods in the art.

Results

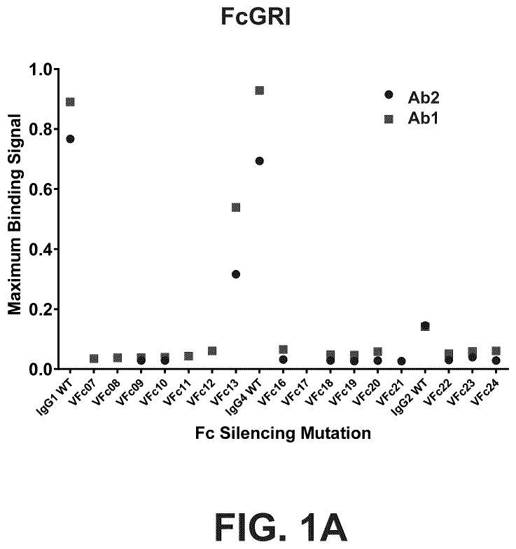

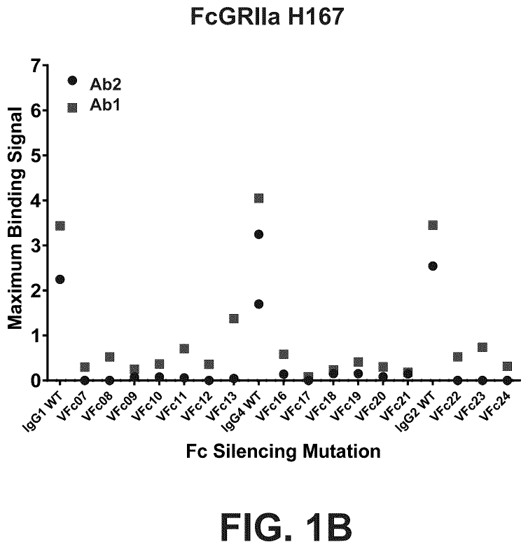

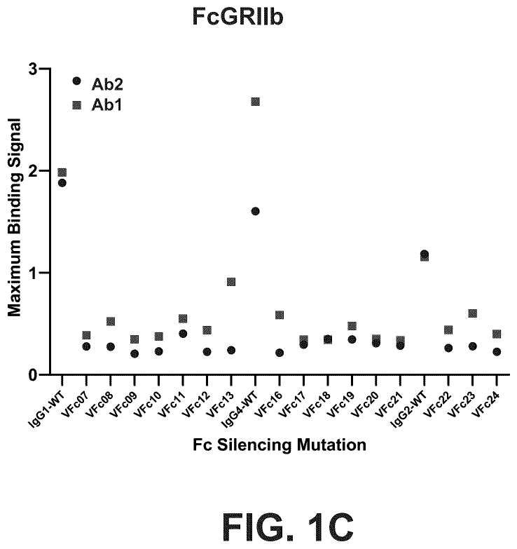

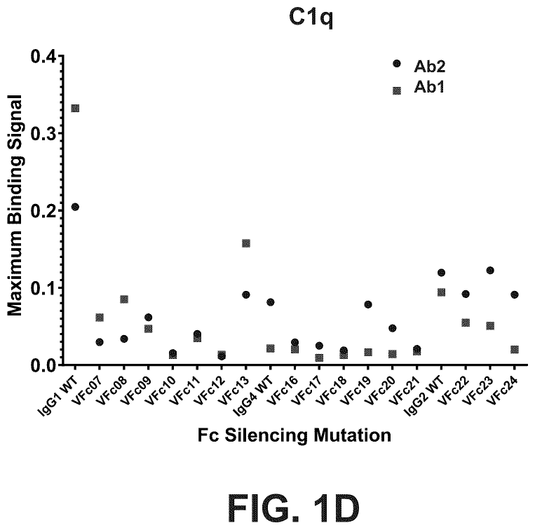

As shown in FIGS. 1A-C, all Fc variants resulted in significantly reduced binding to the FcγRI, FcγRIIa, and FcγRIIb as compared to their counterpart wild-type IgG isotype. Notably, for both Ab1 and Ab2, the Fc region of any one of vFc07-vFc12 and vFc16-vFc24 did not bind to FcγRI, FcγRIIa and FcγRIIb. It was also notable that a single mutation D265G was able to significantly reduce FcγRI, FcγRIIa, and FcγRIIb binding (compare vFc13 vs IgG1 WT in FIGS. 1A-C). Additionally, all Fc variants resulted in significantly reduced binding to the C1q, as compared to their counterpart wild-type IgG isotype (FIG. 1D), regardless of the variable region (Ab1 or Ab2).

### Example 3

### Further Validation of Two Fc Variants in Reducing Fcγ Receptor and C1q Binding

This example validates that the Fc variants of the present invention successfully abolished Fc-Gamma receptor and C1q binding. In this particular example, 2 Fc variants, vFc10 and vFc17 were used in the experiments. vFc10 comprises L234F/L235E/D265G/A330S/P331S mutations in IgG1 isotype, and vFc17 comprises F234V/L235E/D265G mutations in the IgG4-S228P isotype. (S228P mutation reduces Fab-arm exchange by stabilizing the disulfides in the core-hinge of the IgG molecule.) These two Fc variants were each engineered into an antibody “Ab 1”. The engineered antibodies were tested for binding to FcγRI, FcγRIIa, FcγRIIb, FcγRIIIa, FcγRIIIb, and C1q by methods described in Example 2.

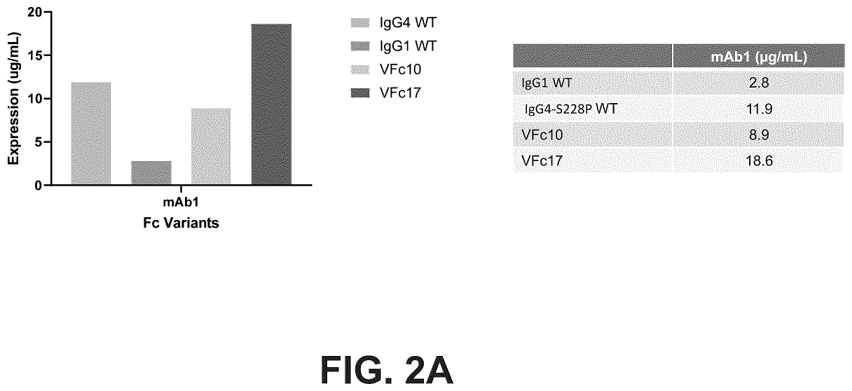

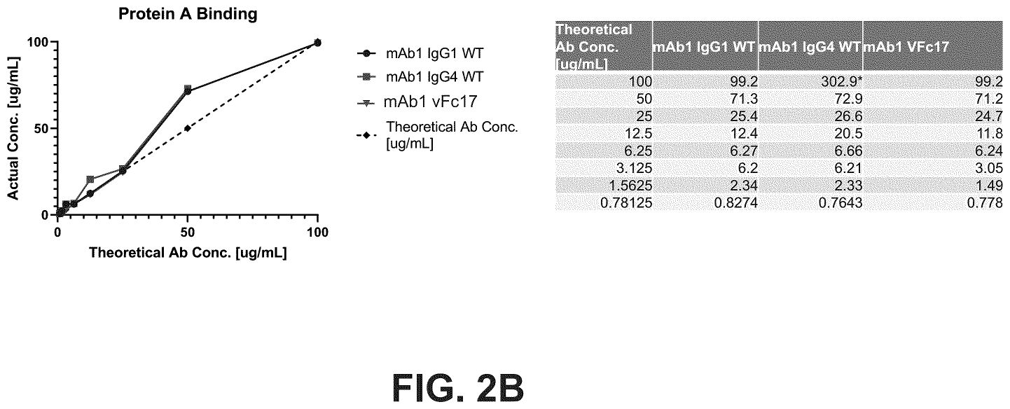

A major shortcoming of antibodies is their demanding production requirements (Garber, 2001, Nat Biotechnol 19:184-185; Dove, 2002, Nat Biotechnol 20:777-779, incorporated by reference). It is therefore important that the engineered antibodies are not limited by expression and purification yield. Wild-type IgG1, wild-type IgG4, vFc10 or vFc17 Fc regions engineered into an antibody Ab1 were expressed in media according to methods known in the art, and the expression yield was measured. As shown in FIG. 2A, both antibodies with vFc10 or vFc17 Fc variants produced high yields, with significantly higher yield than the wild-type IgG1 antibody, and comparable or higher yield than the wild type IgG4 antibody. As one of ordinary skill in the art would appreciate, protein A interacts with the Fc portion of the immunoglobulins. Therefore Octet experiment was performed to check that the mutations introduced in the Fc variants do not alter protein A binding properties. FIG. 2B shows that the Fc variants display the similar binding as the wild-type IgG1 or IgG4, illustrating that the mutations do not alter the protein A binding properties.

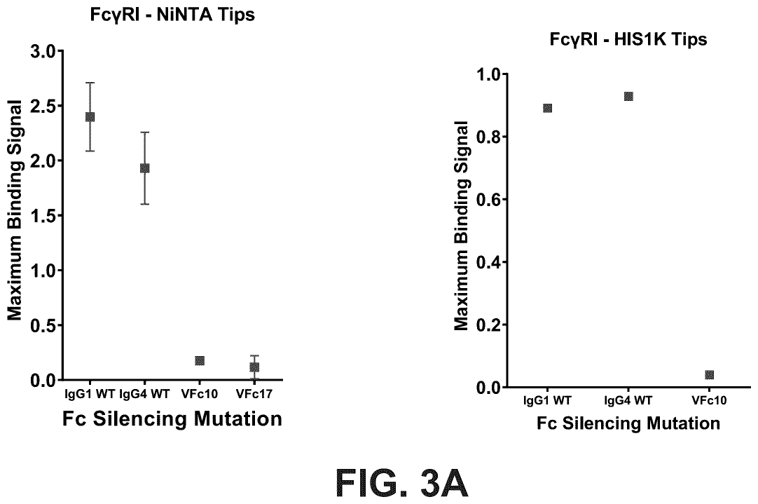

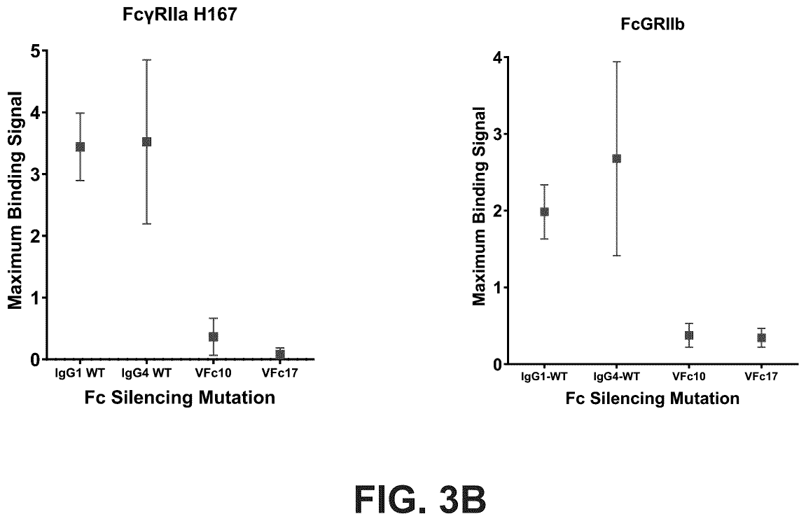

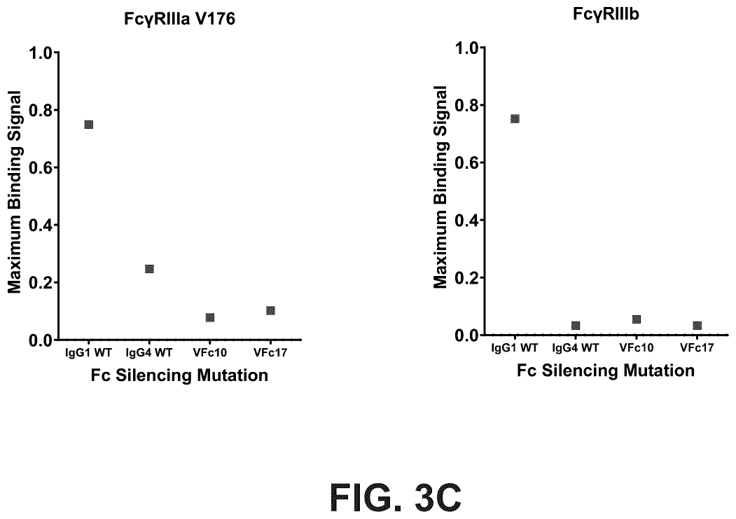

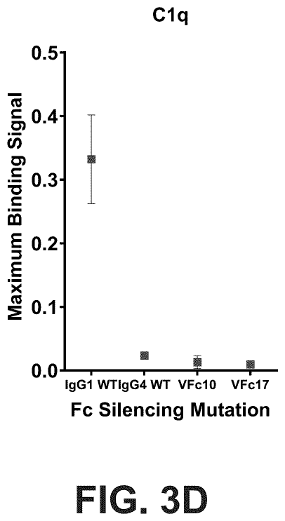

As shown FIG. 3A, both vFc10 and vFc17 abolished FcγRI binding to nearly baseline levels when paired with the antibody. Similarly, both vFc10 and vFc17 abolished FcγRIIa, FcγRIIb, FcγRIIIa, and FcγRIIIb binding to nearly baseline levels (FIG. 3B-3C). IgG4 have short hinge and low Fab arm flexibility which partially shields it from binding to C1q. FIG. 3D shows that neither vFc10 nor vFc17 introduced C1q binding when paired with Ab 1.

Overall, the data in this example shows that the two Fc variants tested showed abolished binding to all FcγRI, FcγRIIa, FcγRIIb, FcγRIIIa, FcγRIIIb, and C1q when paired with a variable region of an antibody.

### Example 4

### Fc Variants Displayed Significantly Reduced ADCC, ADCP, and CDC Induction

Antibodies of the IgG sub-class are bi-functional molecules, possessing a F(ab) domain, variable in sequence and responsible for the binding of antigen, and an Fc domain, constant in sequence and responsible for mediating a range of antibody effector functions. These functions are primarily triggered through interaction with the complement component C1q or with a family of FcγRs expressed, primarily, on the surface of leukocytes. Fc gamma receptors (FcγRs) trigger cell-mediated cytotoxic effector functions such as antibody dependent cellular cytotoxicity (ADCC), phagocytosis (ADCP), and complement-dependent cytotoxicity (CDC).

Antibody dependent cellular cytotoxicity (ADCC) is an Fc-dependent effector function of IgG important for anti-viral immunity and anti-tumor therapies. NK-cell mediated ADCC is mainly triggered through the IgG-Fc-receptor (FcγR) Ma. Phagocytic cells, including monocytes, macrophages, neutrophils, eosinophils and dendritic cells (DCs), express FcγRI, FcγRII, and FcαRI, which can all mediate immune complex uptake. ADCP results in the clearance of immune complexes from the infected host, by trafficking of the complexes to lysosomes for degradation and antigen processing for presentation on Major Histocompatibility Complex (MHC)-molecules on the cell surface. Interestingly, some viruses have exploited this mechanism to infect phagocytes by escaping from lysosomal degradation (described below in “Antibody-dependent enhancement of infection”).

This example shows that the Fc variants of the present invention, which were able to abolish binding to FcγRI, FcγRIIa, FcγRIIb, FcγRIIIa, FcγRIIIb, and C1q, have reduced ADCC, ADCP, and CDC function.

In this particular example, 2 Fc variants, vFc10 and vFc17 were used in the experiments. vFc10 comprises L234F/L235E/D265G/A330S/P331S mutations in IgG1 isotype, and vFc17 comprises F234V/L235E/D265G mutations in the IgG4-S228P isotype. These two Fc variants were each engineered into an antibody “Ab1”. The amount of ADCC, ADCP, and CDC induction was measured against the antibody concentration.

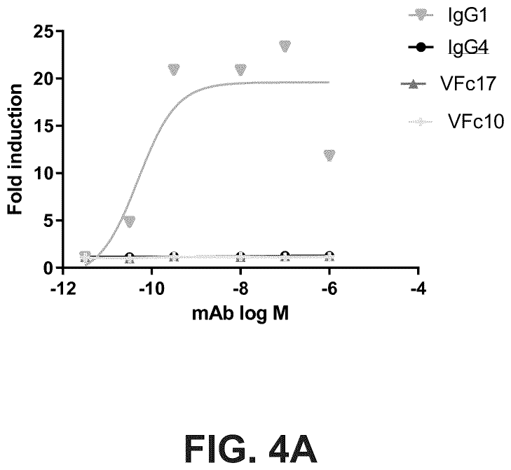

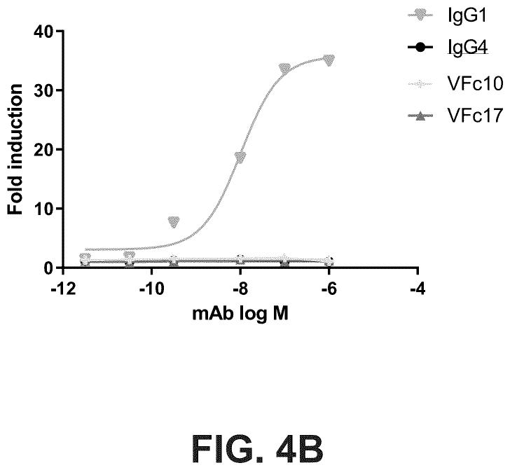

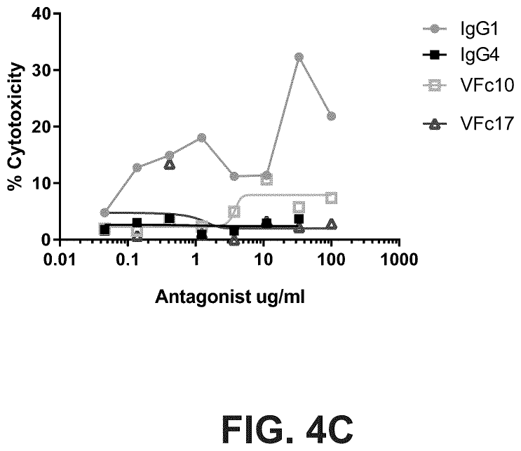

FIG. 4A shows that VFc10 and VFc17 maintained low ADCC as compared to the wild type IgG1 antibody. Similarly, VFc10 and VFc17 maintained low ADCP and CDC as compared to a wild-type IgG1 antibody (FIG. 4B and FIG. 4C).

Overall, the data in this example shows that Fc variants of the present invention abolished binding to all FcγRI, FcγRIIa, FcγRIIb, FcγRIIIa, FcγRIIIb, and C1q, and effectively reduced ADCC, ADCP, and CDC functions.

### EQUIVALENTS AND SCOPE

Those skilled in the art will recognize, or be able to ascertain using no more than routine experimentation, many equivalents to the specific embodiments of the disclosure described herein. The scope of the present disclosure is not intended to be limited to the above description, but rather is as set forth in the following claims:

1. An isolated polypeptide comprising an Fc variant of a wild-type human IgG Fc region, said Fc variant comprising an amino acid substitution at a position selected from 234, 235, or 265, wherein the residues are numbered according to the EU index.

2. The isolated polypeptide of claim 1, wherein the amino acid substitutions are at positions 235 and 265, wherein the amino acid at 265 is substituted to Gly, wherein the residues are numbered according to the EU index.

3. The isolated polypeptide of claim 1, wherein the amino acid substitutions are at positions 234 and 265, wherein the amino acid at 234 is substituted to Val, wherein the residues are numbered according to the EU index.

4. (canceled)

5. An isolated polypeptide comprising an Fc variant of a wild-type human IgG Fc region, said Fc variant comprising an amino acid substitutions of 234V, L235E, and D265G, wherein the residues are numbered according to the EU index.

6. (canceled)

7. The isolated polypeptide of claim 1, wherein the amino acid substitutions are 234F, L235E, and D265G, wherein the residues are numbered according to the EU index.

8. (canceled)

9. The isolated polypeptide of claim 1, wherein the amino acid substitutions are 234F, L235E, G237A, and D265G, wherein the residues are numbered according to the EU index.

10-12. (canceled)

13. The isolated polypeptide of claim 1, wherein the amino acid substitutions are 234F, L235E, D265D, A330S, and P331S, wherein the residues are numbered according to the EU index.

14. (canceled)

15. The isolated polypeptide of claim 1, wherein the amino acid substitutions are 234V, L235A, G237A, and D265G, wherein the residues are numbered according to the EU index.

16-17. (canceled)

18. An isolated polypeptide comprising an Fc variant of a wild-type human IgG Fc region, said Fc variant comprising an amino acid substitutions of 234V, L235A, G237A, D265G, A330S, and P331S wherein the residues are numbered according to the EU index.

19. (canceled)

20. The isolated polypeptide of claim 1, wherein the amino acid substitutions are L235E and D265G, wherein the residues are numbered according to the EU index.

21. (canceled)

22. The isolated polypeptide of claim 1, wherein the amino acid substitutions are L235E, G237A, and D265G, wherein the residues are numbered according to the EU index.

23. (canceled)

24. The isolated polypeptide of claim 1, wherein the amino acid substitutions are L235E, G237A, and P329G, wherein the residues are numbered according to the EU index.

25. (canceled)

26. The isolated polypeptide of claim 1, wherein the amino acid substitutions are L235E, G237A, and L328R, wherein the residues are numbered according to the EU index.

27. (canceled)

28. The isolated polypeptide of claim 1, wherein the amino acid substitutions are D265G, A330S, and P331S, wherein the residues are numbered according to the EU index.

29. (canceled)

30. The isolated polypeptide of claim 1, wherein the amino acid substitutions are 235E, D265G, A330S, and P331S, wherein the residues are numbered according to the EU index.

31. (canceled)

32. The isolated polypeptide of claim 1, wherein the amino acid substitutions are 235E, D265G, and P329G, wherein the residues are numbered according to the EU index.

33. (canceled)

34. The isolated polypeptide of claim 1, wherein the amino acid substitutions are D265G and one or more amino acid substitutions, wherein the residues are numbered according to the EU index.

35. (canceled)

36. A nucleic acid encoding an isolated polypeptide of claim 1.

37. (canceled)

38. A method of making an isolated polypeptide of claim 1, said method comprising; culturing a host cell comprising a nucleic acid encoding the antibody or antigen binding fragment thereof, an culturing the cell under conditions that allow production of the antibody or antigen binding fragment thereof.

39. A method of treating a disease or disorder by administering a therapeutically effective amount of an isolated polypeptide of claim 1.
